(19) United States
(12) Patent Application Publication
(10) Pub. No.: US 2019/0010508 A1
(43) Pub. Date: Jan. 10, 2019

(54) MATING TYPE SWITCH IN YARROWIA LIPOLYTICA

(71) Applicants: Adam G. LAWRENCE, Arlington, MA (US); Peter Louis HOUSTON, Boston, MA (US); Jessica Leigh MCGRATH, Cambridge, MA (US); Joshua TRUEHEART, Somerville, MA (US); DSM IP Assets B.V., Heerlen (NL)

(72) Inventors: Adam G. LAWRENCE, Arlington, MA (US); Peter Louis HOUSTON, Boston, MA (US); Jessica Leigh MCGRATH, Cambridge, MA (US); Joshua TRUEHEART, Somerville, MA (US)

(21) Appl. No.: 16/068,395

(22) PCT Filed: Jan. 6, 2017
(86) PCT No.: PCT/US2017/012462

§ 371 Date: Jul. 6, 2018

Related U.S. Application Data
(60) Provisional application No. 62/276,440, filed on Jan. 8, 2016.

(51) Int. Cl. C12N 15/81; C12N 1/16; C12N 15/10

(52) U.S. Cl. CPC C12N 15/815; C12N 15/1037; C12N 1/16

(57) ABSTRACT

The present invention is directed to a process for switching the mating type of a Yarrowia fungus strain into an opposite mating type, and to the use thereof to sexually cross two individual strains resulting in new strains.

### CROSS REFERENCE TO RELATED APPLICATIONS

This application claims the benefit of the filing date of U.S. Provisional Patent Application No. 62/276,440 filed Jan. 8, 2016, the disclosure of which is hereby incorporated herein by reference.

### FIELD OF THE INVENTION

The present invention is directed to a process for switching the mating type of a Yarrowia fungus strain into an opposite mating type, and to the use thereof to sexually cross two individual strains resulting in new strains.

### BACKGROUND OF THE INVENTION

Yarrowia fungi have been used extensively as a host cell for producing a variety of products. For example, a genetically modified Yarrowia fungus strain was developed to produce high levels of beta-carotene, a natural colorant (U.S. Pat. No. 7,851,199). In another example, a genetically modified Yarrowia fungus strain was developed to produce abienol, a natural fragrance (PCT International Application No. PCT/US2015/063656). Yet, the production level of any product of interest in the initial round of development in Yarrowia fungi strains is often too low for commercial exploitation, rendering substantial strain improvement essential. In Yarrowia fungi, strain improvement is traditionally done by random mutagenesis or targeted gene manipulation using recombinant DNA techniques, followed by screenings for strains possessing advantageous properties. However, this effort is complicated by certain unique characteristics of Yarrowia fungi. In Yarrowia fungi, it is difficult to control the locus where the modified target gene is inserted into its genome. As a result, a number of mutants with various degrees of desired traits may appear but their genetic compositions are unknown. Often, it is desired to combine traits of these improved phenotypes without identifying their genetic causes. However, because the mutants are in haploid form, unless they happen to be of opposite mating type, they cannot be mated to form a diploid strain in order to combine the desired traits. While this problem may be solved by generating mutants in parallel of two Yarrowia fungi strains of opposite mating types, such approach is cumbersome and costly. Therefore, there is a desire to develop a new method to combine traits of improved traits in a more efficient manner.

In 1973, Bassel et al. identified the mating types of Yarrowia fungi, MAT-A and MAT-B (see, J. Bacteriol., 108:609-611). It was further identified by Kurischko, et al (1999) that the MAT-A locus consists of two genes, MATA1 and MATA2 (see, Mol. Gen. Genet., 262:180-188), and by Butler, et al (2005) that the MAT-B locus also consists of two genes, MATB1 and MATB2 (see, PNAS, 10(101):1632-1637).

In 2008, Rosas-Quijano et al. analyzed the role of the MAT-B idiomorph in the mating of Yarrowia lipolytica. He demonstrated that deletion of the MAT-A cassette in an A strain led to loss of mating type capacity in mat-null mutants of Yarrowia lipolytica. He further demonstrated that introduction of the MAT-B locus into the mat-null mutants will create a B type strain.

WO 2011/095374 teaches the use of mating type switch to improve the sexual behavior of filamentous fungus strains. It has disclosed the identification of mating types of Aspergillus niger and Aspergillus tubigensis so as to transform Aspergillus niger into a heterothallic fungus, i.e., filamentous fungus individuals having opposite mating types resulting in one or more pair of strains which two opposite mating types.

However, at the time of the present invention, it remained unclear whether progenies of homozygous A and B strains of Yarrowia lipolytica can be created and whether such approach is a viable one for accelerating genetic trait selection as wished by many Yarrowia geneticists to try in their studies.

It is, therefore, an objective of the invention to provide a process to switch the mating type of a Yarrowia fungus strain to an opposite mating type, and thus allowing the resulting strain to be sexually crossed with another Yarrowia fungus strain so that the desired genetic traits possessed by both strains can be combined in a rapid and efficient fashion.

### BRIEF SUMMARY OF THE INVENTION

The present invention is directed to a process for switching the mating type of a Yarrowia fungus strain to an opposite mating type, wherein an acceptor Yarrowia fungus strain is subject to genetic modification in which one or more mating type locus genes (MAT) of the opposite mating type of the acceptor Yarrowia fungus strain is introduced into the acceptor Yarrowia fungus strain and thus switches the acceptor Yarrowia fungus strain to the opposite mating type.

In one embodiment, the acceptor Yarrowia fungus strain is Yarrowia lipolytica. Preferably, the Yarrowia fungus strain is an industrial strain.

In one embodiment, the acceptor Yarrowia fungus strain has a MAT-B locus in which a MAT-A locus is introduced. In a specific embodiment, the MAT-A locus consists of a MATA1 gene and a MATA2 gene.

In another embodiment, the acceptor Yarrowia fungus strain has a MAT-A locus in which a MAT-B locus is introduced. In another embodiment, the MAT-B locus consists of a MATB1 gene and a MATB2 gene.

The present invention is also directed to a Yarrowia fungus strain obtained by the processes described above.

In one embodiment, the above described Yarrowia fungus strain produces one or more product of interest. In a specific embodiment, said one or more product of interest comprises steviol glycoside, carotenoid or beta-ionone.

The present invention is also directed to a process for producing Yarrowia fungus strain progeny for industrial production, wherein parent Yarrowia fungus strains with two opposite mating types are sexually crossed and their progeny is isolated, and wherein one of the parent strains is generated according to the mating type switch process described above.

The present invention is also directed to a process for selecting a Yarrowia fungus strain with a desired phenotype, wherein a library of progeny produced in accordance with the process described above is screened, and one or more strains with a desired phenotype is selected.

In one embodiment, the desired phenotype is the ability to produce one or more product of interest. In one specific embodiment, the one or more product of interest comprises steviol glycoside, carotenoid or beta-ionone.

The present invention is also directed to a process for the preparation of one or compound of interest, comprising: a. cultivating a progeny Yarrowia fungus strain generated by the process described above under conditions conducive to the production of said compound; and b. recovering said compound of interest from the cultivation medium or cell lysates.

### BRIEF DESCRIPTION OF THE FIGURES

Embodiments of the invention will now be shown, by way of example only, with reference to FIGS. 1-3 in which:

FIG. 1 shows the genetic modifications of ML15186 (boxed in green), leading to strains ML16761 and ML16766 (boxed in blue). Letters in red refer to the treatment/transformations described in Examples 1-9.

FIG. 2 shows homologous replacement of the MAT-B locus with MAT-A locus linked to hygromycin resistance.

FIG. 3 shows the increase in Rebaudio side A production (arbitrary units) following mating procedure.

### BRIEF DESCRIPTION OF THE SEQUENCE LISTINGS

The nucleic acid sequences listed in the accompanying sequence listing are shown using standard letter abbreviation for nucleotide bases. Only one strand of each nucleic acid sequence is shown, but the complementary strand is understood to be included by any reference to the displayed strand. In the accompanying sequence listing:

SEQ ID NO: 1 sets out the DNA sequence of the MAT-A
locus of a Yarrowia lipolytica strain
CAACAGGCCATGGAGGAGGAGCGCATCCGGCAGATGCAGATGCAGCAGCAGCAGC
AGTTTGAAATGCAGCAGCGACAGCAGATGGAAGCGCAACAGCGGGCTCAGGAACA
ACTAATGGCCGACCAGATGGCCCGACATGCCCAGGGTCGAATGGCCGAGCTTGAGC
GAGACATTCTGGCTCTCCGAGGTCAGTACGATCAGGATCAGCTCATGCTGGAGCAGT
ACGATGGTCGAGTAAAGGCTCTGGAGGCTGAGCTGAATCAGCTGCAGCAGACTGCT
CATCAGAGYGCCCAGGCCAAGGATGATCTGATTGAATCTCTTCAGCAGCAGATCAC
CATGTGGCGGCAAAAGTACGAGACTCTGGCCAAACGGTACTCGTCCATGCGAGAGG
AATATCTTGCCTTGCTCAAGAAGCTCAAGGCTACCCAACAGAAGGCTGCGTCAGCTA
AGGAAGCCATTGAGAAGGCTGAGAAGMTGGAGCGAGACATGCGACACAAGAACAT
TGAGCTTGCTGATCTCATCAAGGAGCGAGATCGAGCTCGATATGATCTTGACCGTGC
CAAGGGTGGCAACAAGGAGGACGTGGAGCGTCTGGAACGAGAGCTTCGAATGGCCC
AGGACAAGCTGGCCGATAAGGACCGATCTACCGGTGCTGATCTGTCTCTTCTTTTGT
CGAAGCATAACCGAGAGCTTTCAGAGCTTGAGAATGCTTTGAAGATGAAGCAGCGG
GCTCTGGACGAGCGAGGAGATGACTCTGATCTGTTGAGACGACTGGAAGAGAAGGA
GATTGAGTACGAGGCTCTCAACGAAGCCTTCAACTCGCTGGCTCTGCACCAGGAGC
AGCAGGGCAACAACAGCAACGGTGGATCCACTCCCCTGGCTGCTTTGCATTCTATCA
TTGATGCTCTTCTAGAGTCCGGTTCCCAGCGTGTTCAGGACGCTCTCTTCGAGCTCGA
GTCGCCTATGCAGGCCGGTAACCAGAACTCGACTCCCGAGTATCTCTTGTCTGTCAT
CGAGAAGGCGTCCGAGTCTGCTTCCGCCTTTGCCACGTCGTTCAACAACTTCTTGGC
CGACGAAACCGATGGCGACTACGCCGAGATCATCAAGACCGTCAACATCTACTCTA
CTGCTGTGGAGAATGTTCTGTCCAACTCCAAGGGTCTCACTCGTCTGGCCAAGGACG
ATGCTTCTGCCGACGCCCTTGTCAACTTTGCTCGTGAGTCTGCCGAGGCCACAGAGC
GAAACTTTATTGGTCTTCTGTCTGAGATCATTGAGGATTTCCCTCTGGATGACAAGAT
GGAGCGGGTCATCACTCTCAACATGAACGTGCAGACTGCTCTTCAGCACCTCACCAA
GCTGGCCGAGCGAATGGCCCCCAAGACCAACATCAACATGTCTGGTGATCTGGGCG
ACCTCGTTGAGCGAGAGATGGCCAAGGCTGCTGATGCCATTNNNGCTGCTGCTTCTG
CTAAGCTTGGCGATCTGCTCAAGTTTAACCAGTCCGACCCTCTCAAGTCGACCACCG
ACCTGCAGCTGCACGAGGCTGCCATCCAGGCTGCCCAGGCCGTCATCAACGCCATTG
CTGCGCTGATTCGAGCTGCCACCGATGCCCAGAACGAGATTGTAGCCCAGGGACGA
GGTACTTCTTCTCGAGCACAGTTCTACAAGAAGAACAACAAGTGGACCGAGGGTCTT
ATTTCCGCGGCCAAGAGTGTAGCTGCATCGACCAACATTCTCATCGAGAAGGCCGA
CGGCACTCTCCGACGAACCTCTGGTCTCGAGGAGCTCATTGTGGCTTCCAACAACGT
CGCTGCTTCCACTGCTCAGCTGGTTGCTGCCTCTCGAGTAAAGGCTACCTTCATGTCC
AAGACCCAGGACAAGCTGGAAGAGTGTTCAAAGGTGGTTACTTCCGCCTGCCGAAA
CCTCGTCAAGCAGGTGCAGGAAATTCTCAACAAGAAGTTTGGCGAGCTGGACGAAA
AGGTAGACTACGCTGCCCTCTCCAAGCACGAGTTCAAGACGACCGAAATGGAACAG
CAGGTCGAGATTCTCAAGCTCGAAAACGATCTGCAGGGCGCCCGAAAGCGACTTGG
ACAGATGCGAAAGGTCGCCTACCTGCATCAGGACGAGGAGGAGTCCATTCCTGGCC
AGGAGGACTAAGCTACGCGCGGCTGATGTATGTATAACATGTATGGTAAATGAATG
AAATGATATTTTTAGTGATCGAATGATGAAGAAGGCTCTGCGATGCTTTACTCGTTA
CATGACAACTGTCCAATGCGACAGTTCTATGTTACTATGACACACACTATTTATTAC
GATGCAAATTATGTTGACATGCATGAGACAAACCCCACTGTAACCACTTTATTAGAA
ATGCTAAGGGCTCTCAGGGAAGCTGCACTCATCAATGGACCCGGGACTAGTCATGG
GTGAAGGTATGAACCTAGTTGATCCATCATCAGGGGAATCCGTCTTGGGCTCTCTGA
ACCGACGATTGGTGAACCATTGTGTAACCTGATGAAGTGAGATTCCACAATGCTCTC
CAATAATTCTCCTCTCTGTTCTGGTTGGATGAGAACAGATCTTGAACAGAGAGTCAA
GATAGGCGGTAACCTCGTTACTTAACCTATTCCGGACTTTCTTAGGAAAGACGTCAC
AGTTGTGATCCAGACTCTGCAACTTGTCAGCTTCTTTTAGAGCTCTGGCTTTGACTTC
GAGGTCTTTGGTGCTGGCCTCATAGGCTCTGATTTCGCCTCTATGTCTTTGTCTAAAT
TTGCTGCGACCTCTAGATAGTCATCAAGCAGCTCTGCGGCAATTCCTTTTGCAAGAC
TGTACTCATCATTCCAATCAGCACCCAGTATGGCCTTACTGGTCATCCATTCGTTCTG
TTTTAGTATTCCCCAATACTGCTCTTGTTTTCAATATACTCACTAATCTCGCTGGTAC
CAGTTGCTGGATGTAAGTGATGTAGTGGCGAATCAGCCTGTCGTATTTGATTCTGTG
TTCATTTGGAACACCCTCCAAAAAGGTCCAAATCGTTGTGCTTATGTAGAGTCCACT
CTCAGGGAATTTGGACATCCACTCAGAAATCTCTTCAAACCATTTAAGGGAACTGGA
GATTTCGATTGACTGTTCGTGAAGGGCCTGGCACGTGGTTTCGCCTTTTCTTGCGGCC
AAGATCAGGCGCTGGCAGCGCCAGATATCTGTAGGGGTTCGGCTAGGCATGTGATG
TGGGGAGAAAAAAAATGTCGTATCTGTTTAATTAAGTACCAGGGTGTACTTGGTGGG
GCCGGTTTCTAGATATATATTTACAATGGTCACTTGGACTGGTCAGACGAGGTGGAT
GTGGCTAGTTTACCCCGATATTATTCATGCACGCCGTGCATATGGTTCTGGAACAAA
GACACCTTAAGCGACATTGGCTCTTGCCTCATCACAATTGCCTAAACTAGAGATCTA
GTATTTCCAGACCAACACATATCGACAAATATATATAAACAACATGCAACTCTCTTG
GCTGGCTATTATCAGCTATACCAAGCCACACATAACATCCTCAAAATGGAAAACAC
GATACTACACATTCATTCCTTTCAACTACCCCAAACAGAACAGCCCTACCCCGAGGC
TATGTTATTCGACAGGGACACTAGCGATTCACGTACGGTTCTAACTCAGAAGCCAAA
TGGACTGGAAATATCACTCAACTTTTTGCAATATGACGGTCACAAGGGGCTGTTCAT
CAGGCAAGACAGTCGAACGAATGAGCCTGAGTACATTGAACCCAAAGTTCTGAAAC
CTAGGAACAGCTACATCTTGTTCCGAAATGCAACATCCAGATGCTCTCAGAGCATTG
ATCCCAACTCAGCATTTGTTTCGAGGGTCTCCAGTTATATCTGGGGCTCGGGAATCC
CCAATCCTATTGTTCGGCGATGGTTCAAGTATATGGGTTTCTTCGAAGCCCTATACCA
CGAAGTCGACTATCCTGAGTATATCCCGAACAAACTGCCCAAGTCCCCAAAGAAGC
CGAAGGTCCAGAAGGGTGCAACTAAGTCTAAGAAAAAGGGAGCAAAGGGTAAGAA
CAAGGTTACTGCTCTCCAAGTACTGGTCGGCCCTACCGTTGGTGTCGGGGGAAGGAT
GACTGTTAGCCCAATGACTGCTTTCTTTAGCAACAACTCTCACGTTACGTGCTATGAC
CCCAACTTTGTTCTCGATACACCTACTCGAGAATTCCTCATGATGCCCCTGGATGATA
TCACTCTCCCATCGCAAGGACCAAGATCAGATTCACAGGAGCAGCACGTCCCTCGG
CAGCCTCCCGATGGGCAGGACTATTTTGACATTCTGGATATTGATGATTTCGTTTCTC
CTTATGATGACTTGACTACTCGCTCGTTCACCAACAGGCAGCTTTTTAAGCATGCCA
CACTGGGTTGAGCTTGGTAATTTTCTGTACTCACTGTACGTTTATCATTGGACACACT
AACGGTATTATAATTTAATTTTATTTCCACTGAAAGAAGTTACATCTGGCACTGGTGT
CGTCCTGGGAGCCCTTGGCGTCGGGGTCTGCCGATGCCTTGTAGGAGTCGCCGACGA
GCTGCAGCGAGTGGTTCTGGCCACAAACCCAAAAGTGCTTGCCCTGGTTGGGTCCCT
TTTTGCGGGTGGTTCTTAGCACCGCCGGGAGGTTGTGGGCACAGACGGGGGCTGGTT
TTTTTGTCATGATCTTGGACCACTGCGACGCGACCTTTTCGGCCTCCTCCACGCGCTC
CGCAATGTCTAGGTACTCGGTGGGCTCTGGCAAGTCCAGTCTGGTGGCCATTTGCGG
TCTCGTATCTTCACCCTCTCGAGTCGCATGCGCACCTGCATCGCAACCTTGATAGGT
GCTTTCAATATTTGGGGACACGGCGGGTGATTGTGATTGTCCCAACGGCTTTGTGTG
CACATCCGAGACGCATGTTGATACGACGGCAGCTTCTGGATCTGGCTTTTGGAAGAA
GGAGGAGATGGCCGCCTGTTTTTTGGCAGTCGATTTGGAGGGCGCTTTGGGTTTGGT
AATGCCCTTGGTCGGCGGCGTTTGCGCCCGGAACATGGACATGACCGAGGTGGTCTG
CACAAACTGCTTGTTAGTTCTGTGGAACAGTTTTGGCCGAAACGAGTTTGGGGCTGA
GGACGACGCTTGTTTGGTCTGGTTAGCTGCACCTTGAGTGGCACGGTTGTCTTTTTCT
TGGCACGCGTCTGGTTCGCGCACCTCTGCATAAACGGGACAATGGTCTGAGCCCTCC
AGGTGGGGCAGAATATCGGCATCCTCCACCTTCAGACCTTTTGAGGCCAGCACGTAG
TCGATACGAGACCCAAAGTTTCCGGGCCGGTAGTTCATCATCACGTTCCAGCAAGTA
TACATGTCGGGTCGATTGGGATGCTTGTTGCGGCACGAGTCCTGTAACAGGTCGGGG
ACCAGTCTGTGGAAGACTCGTCGGCCAACTTTGGCTTCCCTCCAGCTCTTACACTGT
CCCGGGTTGAGCTTTTCAAACACCTCCACAAACCTGCCCTCCTCCTTATCTCCCAGTG
TCGGTAGAGTGATGTGTTTGGCCTTGTGCAGCGCTTGCATTCCCTCAGCTGAGTCGT
ACAGCTCGCGTGCCACATTGAGATCTCCCATGACCACCACCTCTCGTCCAGCCTCAA
CCAGAAGGTTGACCCGTTCCTCCAGCAACCCAAAATAGTCGTCTCTGTAGGCATCTC
GCTCCTCGCCCTCGGTGGCGGTGCTAGGACAGTAGAGCCCAAACACGACACAGAAC
CCCAGGTCCAACACGAGAGACCGGCCCTCCGAATCGACCTGAAAGAGCCGCTGTGG
ATTGCCCTCGGGATAGGCGCCGATACAACTCTTGCTGCTGCCGTCTCTCTCCCTTTGC
CGCTCGTACAGCTGCTGGTAGGTGAGTCCCTTTTTGGAGTCCGGAGATACCAGCTGA
CCGGTAAGACCCTCCTCGGCCTTGAG
SEQ ID NO: 2 sets out the DNA sequence of the MAT-B
locus of a Yarrowia lipolytica strain
ATGAGATTCGTCACGTTCAACATCTGTGGCATCAACAACGTGCTGAGCTACCATCCA
TGGAACGAGCAAAGGTCGTTTGAGCACATGTTTGCGGTGCTGAAGGCGGACGTGAT
CTGTTTACAAGAAACCAAGCTCCAGCCCCATTTGCTGAAGCGAGAACACGCCCTTGT
GCCCGGATACGATTCGTACTGGTCTTTTTCCAACACAAAAAAGGGCTACAGCGGCGT
GGCGGTCTATGTGAAACATGGCATCAAAGTTCTCAAGGCCGAGGAGGGTCTTACCG
GTCAGCTGGTATCTCCGGACTCCAAAAAGGGACTCACCTACCAGCAGCTGTACGAG
CGGCAAAGGGAGAGAGACGGCAGCAGCAAGAGTTGTATCGGCGCCTATCCCGAGG
GCAATCCACAGCGGCTCTTTCAGGTCGATTCGGAGGGCCGGTCTCTCGTGTTGGACC
TGGGGTTCTGTGTCGTGTTTGGGCTCTACTGTCCTAGCACCGCCACCGAGGGCGAGG
AGCGAGATGCCTACAGAGACGACTATTTTGGGTTGCTGGAGGAACGGGTCAACCTT
CTGGTTGAGGCTGGACGAGAGGTGGTGGTCATGGGAGATCTCAATGTGGCACGCGA
GCTGTACGACTCAGCTGAGGGAATGCAAGCGCTGCACAAGGCCAAACACATCACTC
TACCGACACTGGGAGATAAGGAGGAGGGCAGGTTTGTGGAGGTGTTTGAAAAGCTC
AACCCGGGACAGTGTAAGAGCTGGAGGGAAGCCAAAGTTGGCCGACGAGTCTTCCA
CAGACTGGTCCCCGACCTGTTACAGGACTCGTGCCGCAACAAGCATCCCAATCGACC
CGACATGTATACTTGCTGGAACGTGATGATGAACTACCGGCCCGGAAACTTTGGGTC
TCGTATCGACTACGTGCTGGCCTCAAAAGGTCTGAAGGTGGAGGATGCCGATATTCT
GCCCCACCTGGAGGGCTCAGACCATTGTCCCGTTTATGCAGAGGTGCGCGAACCAG
ACGCGTGCCAAGAAAAAGACAACCGTGCCACTCAAGGTGCAGCTAACCAGACCAAA
CAAGCGTCGTCCTCAGCCCCAAACTCGTTTCGGCCAAAACTGTTCCACAGAACTAAC
AAGCAGTTTGTGCAGACCACCTCGGTCATGTCCATGTTCCGGGCGCAAACGCCGCCG
ACCAAGGGCATTACCAAACCCAAAGCGCCCTCCAAATCGACTGCCAAAAAACAGGC
GGCCATCTCCTCCTTCTTCCAAAAGCCAGATCCAGAAGCTGCCGTCGTATCAACATG
CGTCTCGGATGTGCACACAAAGCCGTTGGGACAATCACAATCACCCGCCGTGTCCCC
AAATATTGAAAGCACCTATCAAGGTTGCGATGCAGGTGCGCATGCGACTCGAGAGG
GTGAAGATACGAGACCGCAAATGGCCACCAGACTGGACTTGCCAGAGCCCACCGAG
TACCTAGACATTGCGGAGCGCGTGGAGGAGGCCGAAAAGGTCGCGTCGCAGTGGTC
CAAGATCATGACAAAAAAACCAGCCCCCGTCTGTGCCCACAACCTCCCGGCGGTGC
TAAGAACCACCCGCAAAAAGGGACCCAACCAGGGCAAGCACTTTTGGGTTTGTGGC
CAGAACCACTCGCTGCAGCTCGTCGGCGACTCCTACAAGGCATCGGCAGACCCCGA
CGCCAAGGGCTCCCAGGACGACACCAGTGCCAGATGTAACTTCTTTCAATGGAAAT
GATCGAAGGTTGGCTATTGTAACAAGGAAACTGCCATTTCAGATGTTTACAACTGCT
TTTGATATATTATACTTTTATAAAAGATGAGGATCGACAACGATAATGCACAATGAG
CCTGTTTCACCAACACTGTCATCCCATTTGGGTTAAGTCAATATCAGCGTTCCTATTA
CCTAAAACCGAAGGGTCTACTACTTATATAGTATTGGTAGTTTCTTTGACACGTCCA
CTCTCGTCTTACATATCATACCACTACCGAATGAAGGAAACCAGAGATCTTCTCTTC
TACAACGAGAACTCCATCGACTTCAACTTCTCTCCAGAAGAGATGGACACCCATCAA
ATGATGTGTGTCAACAATGACAGCCAACAGGACACATCGTTCCTCACAGCTACTACT
TGCATTTTTGACTTCCAGAAGGACATCTATGACGACCAAAACCTGATGTCCTTCGAC
CCCTTCTTTCCACAGCAGCGAGATATGGCTTTGTGGATTGCTTCAGTGGGAAATGGG
GCTATCGATTGGAGTACTAAACCTCAACTGACTCTTTCAAAGAAGATCCCTGACGTC
GTCAACGAAAAAGGAGAGGAGATAACCCCTGCTGACCTACAACGCCACTATGCCGA
TAAGCATGTCGCCAAACTTCCAGATCATTCCTGCGATCTTTACTACCTTCTATTCAAG
CATGCCAAATTCGAGGCTACCACCCTGAGGATCCCATTGTCACTCCTCAATGACAAC
GGTACAGGAGCACTTGGCCCATTCGACGAGATCAGATCGTTGACGGATGAAGAACA
ACGTCTTCTTACTCTGCTTAAGACAATTTCACATTACAGCTGGGTCACTAAACATGC
GACCAAAGACTGTACGGGCCAACTGCTTGACCTGTGCAAAGAGGGCCTCCAGCTTG
TGGAATCTATCCAGGTTAACAAAACTTTCAACGGTGTCATTCTGCATATCCTAGTCG
CCTACTTTGGGAGAAGATCTACTGCTCTAAGAGATGAGTTTCAACTGCACAAGCTCT
TTCTTGAACAACGGCTTGACATTGAGTTGGAAATCTTGGCCCAACATGGAGGCGCTG
ACGCCATTGATTCTGAAAAGCTGCGGAAGGTCAAGAAACTGAGCGAACTGTGGGAG
TGGTTCAATGCCATCCCGTACAACCACCACCCTAGCGTCTCTCAGATCGAGTTTATC
GCTTCTCAGATCGGGGAGAAAGTAAGTTTCGTGAAATCCTGGGTTGCAAACAAGCG
ACGAACTGGGGCCAAGAAGAGATCCAAGAAACCGGCACCTTCGACACAGATTAGAC
CTGCATTGAAGGTATTCTTGGAGAAGAAGCCATTTGTGTCTCGCCAAGTTGTGTAAT
TGAATCATAATGTATTACAATGTTGTTAGGTACATACTGTAGATTGTTTTATATGCAA
TATTGTGCCCAATACACAAGGTACAGTATGTACAATACAATACCTAATGTTCATACT
GAACACCTTATATACAAGTACATTAAGGTAGTTGTATGTATGTACATACTGTAGTCA
CTACAGACGCTATCTTTCCAGTGTGCCGACCGGTAACTGAAAGCAAATATAGAAGTA
CGGTTTAAGAAAGATATCATTTAATCAAAGGTGCGATATCTAGAGGCTTGCCCCATT
CTTTGTTTGTGCAATGAACAGGGAACATGCTATGCAGTAACACCAAATAATGTACTT
AGTCTAATCACCTTCTGTTATATTTAGTTATCTTCGCTCCTCGTCTTATTGTCGTCAAT
CTAAGTAGGTACGGTCGCCGCAAAAGGAAATCTCTTGCCGCCGTCGCTACAGTGATC
AAGTAGCTGAGCTACGAAGGTAATTAAAATATTTGTTCGCTTTGCTCATACGTCGTT
TTAATCTTAAATTTCATCTGATTACACTTCGGCGAGGAGTTGTTTCTGGCATCTCGCC
CTTCGACCTTGCTTATGATTTCTGTGACCAGAAACCTAAATTACCCACAATGTGAGTT
GCAAATGCAGGCAGCTAATTACAGTACAGTACAGTACAGTACTGAACTGTATGTAC
AGTAGGTCACGAAGAAGTTAATACTTGTGAGGCAGAGTACCACTTCACAGGGATGC
AATTGCAATGTAGAAAAATACTGTGAAAGTAAAGTATTAAACCTATCCAAGCAAAA
TAAAAGTGTTGACTCAAATAATTCAACCCTTTCCTACCAGCAAGCAATGGAGAAGTA
AAGACGAGGGACTGCCACCCATCCTCAAGCTGTTACTCAAGGTTTGAAAAAGAGTT
CGGACACTCAACTTCCCCAGCCAAAATCGCATAAACAAAGAATCTCGCGAGATTGG
GTTTATCACCTAATGAGGTAATCCTCACGATGAGGCAAGATGAGTATCATATAAAGC
AAAGGATTTTTTCAATCTCAAAAGCAGCTATAACAATGATCAAGATAAAAACCGAG
CACAAAGTGGTGAAGGCAAGAACCCCGAAAAAAATGAAACCTGGAATGGCCCGTTT
CAGAATTCAAGCCGTTCCCATCATCAACCAAAACAACAACGCCTACCTGTCAGAGA
TCAGCTTCGAAGAAAAGTTAGAACCAGTTCCGCCGATGTATCCTGAACTACAAAAA
GTCCTTGATTACATTCACTCACGCAAAGTCCCCTTCGACGGATCATCGGGGCCATAC
CCTCCCTCGAGCAGACGACAAGTGGTCAACGGTTATGTCGCATTTAAGCTATATCAC
CACGTTCCACACACTGACCTTAGCGCAATTGATATCACCCACCTCCTCCAAGGTCCT
TGGAGAGCTTGCCCTGATAGACACATCTGGACCAAGTACGCTGATCACTACCGTTCT
AGAGGGCGTGACGTCGCATTCAAGACATGGCTTCTGTCTGTTACTAGTCCTGCTCCT
GAGAAGGGGCTAATTATTCAGCCTCAAACTCAGAGCGAGCAGGGGTACAACTTCTC
ATCTTCCTCTGATTTTTCAAGTTCTCCAGAACACAGTGAAACACTCCCTGTGTCATCT
GGAGAGCCCAGTACACCTGTTGACCCCTTCCTGATCGAGTTTGATCAAGGCATCGCG
GAGAAAGAGGCCAGCTTCACAAGTCCTTGCAACCAAAACTATGACTTGGATTTCAG
CCAGCTTCAGCCTCTAAGTATTCGCGTCCCTTATTTTAACCAGTACGCTTCTTCCAGT
AACTCTGGATTGGATGTTGACTACTTTGACGGTAGCACCCTAGACACTCCTAGCTAC
ATCGACACTTTCATCACACCATTGCACTAGTCATAATGGTGTAGACATGGGTTTGTA
CGAGTACTACGATACGACCTCAAACTCCAACGGTAGTCCTATTTATTCACATCGTTA
ATATCATTTCATTCATTTACCATACATGTTATACATACATCAGCCGCGCGTAGCTTAG
TCCTCCTGGCCAGGAATGGACTCCTCCTCGTCCTGATGCAGGTAGGCGACCTTTCGC
ATCTGTCCAAGTCGCTTTCGGGCGCCCTGCAGATCGTTTTCGAGCTTGAGAATTTCG
ACCTGCTGTTCCATTTCGGTCGTCTTGAACTCGTGCTTGGAGAGGGCAGCGTAGTCT
ACCTTTTCGTCCAGCTCGCCAAACTTCTTGTTGAGAATTTCCTGCACCTGCTTGACGA
GGTTTCGGCAGGCGGAAGTAACCACCTTTGAACACTCTTCCAGCTTGTCCTGGGTCT
TGGACATGAAGGTAGCCTTTACTCGAGAGGCAGCAACCAGCTGAGCAGTGGAAGCA
GCGACGTTGTTGGAAGCCACAATGAGCTCCTCGAGACCAGAGGTTCGTCGGAGAGT
GCCGTCGGCCTTCTCGATGAGAATGTTGGTCGATGCAGCTACACTCTTGGCCGCGGA
AATAAGACCCTCGGTCCATTTGTTGTTCTTCTTGTAGAACTGTGCTCGAGAAGAAGT
ACCTCGTCCCTGGGCTACAATCTCGTTCTGGGCATCGGTGGCAGCTCGAATCAGCGC
AGCAATGGCGTTGATGACGGCCTGGGCAGCCTGGATGGCAGCCTCGTGCAGCTGCA
GGTCGGTGGTCGACTTGAGAGGGTCGGACTGGTTAAACTTGAGCAGATCGCCAAGC
TTAGCAGAAGCAGCAGCAATGGCATCAGCAGCCTTGGCCATCTCTCGCTCAACGAG
GTCGCCCAGATCACCAGACATGTTGATGTTGGTCTTGGGGGCCATTCGCTCGGCCAG
CTTGGTGAGGTGCTGAAGAGCAGTCTGCACGTTCATGTTGAGAGTGATGACCCGCTC
CATCTTGTCATCCAGAGGGAAATCCTCAATGATCTCAGACAGAAGACCAATAAAGTT
TCGCTCTGTGGCCTCGGCAGACTCACGAGCAAAGTTGACAAGGGCGTCGGCAGAAG
CATCGTCCTTGGCCAGACGAGTGAGACCCTTGGAGTTGGACAGAACATTCTCCACAG
CAGTAGAGTAGATGTTGACGGTCTTGATGATCTCGGCGTAGTCGCCATCGGTTTCGT
CGGCCAAGAAGTTGTTGAACGACGTGGCAAAGGCGGAAGCAGACTCGGACGCCTTC
TCGATGACAGACAAGAGATACTCGGGAGTCGAGTTCTGGTTACCGGCCTGCATAGG
CGACTCGAGCTCGAAGAGAGCGTCCTGAACACGCTGGGAACCGGACTCTAGAAGAG
CATCAATGATAGAATGCAAAGCAGCCAGGGGAGTGGATCCACCGTTGCTGTTGTTG
CCCTGCTGCTCCTGGTGCAGAGCCAGCGAGTTGAAGGCTTCGTTGAGAGCCTCGTAC
TCAATCTCCTTCTCTTCCAGTCGTCTCAACAGATCAGAGTCATCTCCTCGCTCGTCCA
GAGCCCGCTGCTTCATCTTCAAAGCATTCTCAAGCTCTGAAAGCTCTCGGTTATGCTT
CGACAAAAGAAGAGACAGATCAGCACCGGTAGATCGGTCCTTATCGGCCAGCTTGT
CCTGGGCCATTCGAAGCTCTCGTTCCAGACGCTCCACGTCCTCCTTGTTGCCACCCTT
GGCACGGTCAAGATCATATCGAGCTCGATCTCGCTCCTTGATGAGATCAGCAAGCTC
AATGTTCTTGTGTCGCATGTCTCGCTCCAGCTTCTCAGCCTTCTCAATGGCTTCCTTA
GCTGACGCAGCCTTCTGTTGGGTAGCCTTGAGCTTCTTGAGCAAGGCAAGATATTCC
TCTCGCATGGACGAGTACCGTTTGGCCAGAGTCTCGTACTTTTGCCGCCACATGGTG
ATCTGCTGCTGAAGAGATTCAATCAGATCATCCTTGGCCTGGGCGCTCTGATGAGCA
GTCTGCTGCAGCTGATTCAGCTCAGCCTCCAGAGCCTTTACTCGACCATCGTACTGC
TCCAGCATGAGCTGATCCTGATCGTACTGACCTCGGAGAGCCAGAATGTCTCGCTCA
AGCTCGGCCATTCGACCCTGGGCATGTCGGGCCATCTGGTCGGCCATTAGTTGTTCC
TGAGCCCGCTGTTGCGCTTCCATCTGCTGTCGCTGCTGCATTTCAAACTGCTGCTGCT
GCTGCATCTGCATCTGCCGGATGCGCTCCTCCTCCATGGCCTGTTGCTGCCGTAGCC
GCTCCTGCTCGGCGTCGTACTGTTGCTGGGCGAGCAGCGCGTCCTGGGACCAAAAGT
TCTCAATCGGCTGAGCCTCAGCAGGAATAGAGGGCGTAGGAGTCGCCACAGGCAAG
GGAGCGGGCTCTGGAGACACAGATCGGGTCTCGGCCACGGACTTGGGTCGCTGGGG
GAGTCCAGGTCCGTCCTCCTCTTCAAGCAGGTTGGGAGGGTTCTTGCCGAGCTGCGG
GATAGTCACAATGGATCGCAGGTACTTGAGAGTAGAGCAATCGTAGTAAAAGTCTC
TCAGTCGGTCGTGCTGCGAGTAGAATCGCTGCCGGAGAGACTCAAGAGCCTCGTCGT
TACCAGTAGACACATGCATGGCTCGCAGCATGGAGATGACGAACCGGTAGATTCCG
TACGACTCGGCAATCATGGGAACCAGAGACGAGATTTTGCACTCGTTGGATCGGGA
TCGCTGCAGCGACGAGAAGACAAGTCGCTGGAAGTCGTCCACTCCGTCCTGGAGGG
TCATCAGGTTCATAATGGCCTCGTATCCCTCGTTGGGGTCGTTAACGGTTCGCAGAG
AAATGTAATCCTCGTACTCAAACATTCCGTTGAAGGCCGGGTGGTACTTGTGGAAGG
TGAGCTTCTGCATGAGGAACCGGACATACTCGGAGATGAGCTTGCCATAACCGTGGT
TGCCCTGGTTAGGACCAGTGTAGTTGTTTCCTCGAGCCAACGACTGGATGAAGTTGA
CATTGGCCTGGGCATCTCTCAGCGCCGATGGGTGGCCCTCCTGAAGCACCTTGTGGA
TGGTGATCAGGGCCTTGAAGACCATGACATCGTCGGTCTGCAGGGGCTGAATTCGA
ATGCCGTTCCAGAAGGCCTTGGACGACCGGTGGTCATGGGTGTACACAATGCATGCT
CGCACGTGTTTCCGCTTGGGCGCAGACTCGTCTGTGTTGGTCGCCTTTTTGATGTTGA
CGTTGAGGTCCACTTCGGCTCTGTTGGAAGACAT
SEQ ID NO: 3 sets out the DNA sequence of the MATA1
gene of a Yarrowia lipolytica strain
ATGCCTAGCCGAACCCCTACAGATATCTGGCGCTGCCAGCGCCTGATCTTGGCCGCA
AGAAAAGGCGAAACCACGTGCCAGGCCCTTCACGAACAGTCAATCGAAATCTCCAG
TTCCCTTAAATGGTTTGAAGAGATTTCTGAGTGGATGTCCAAATTCCCTGAGAGTGG
ACTCTACATAAGCACAACGATTTGGACCTTTTTGGAGGGTGTTCCAAATGAACACAG
AATCAAATACGACAGGCTGATTCGCCACTACATCACTTACATCCAGCAACTGGTACC
AGCGAGATTAGTGAGTATATTGAAAACAAGAGCAGTATTGGGGAATACTAAAACAG
AACGAATGGATGACCAGTAAGGCCATACTGGGTGCTGATTGGAATGATGAGTACAG
TCTTGCAAAAGGAATTGCCGCAGAGCTGCTTGATGACTATCTAGAGGTCGCAGCAA
ATTTAGACAAAGACATAGAGGCGAAATCAGAGGCCTATGAGGCCAGCACCAAAGAC
CTCGAAGTCAAAGCCAGAGCTCTAAAAGAAGCTGACAAGTTGCAGAGTCTGGATCA
CAACTGTGACGTCTTTCCTAAGAAAGTCCGGAATAGGTTAAGTAACGAGGTTACCGC
CTATCTTGACTCTCTGTTCAAGATCTGTTCTCATCCAACCAGAACAGAGAGGAGAAT
TATTGGAGAGCATTGTGGAATCTCACTTCATCAGGTTACACAATGGTTCACCAATCG
TCGGTTCAGAGAGCCCAAGACGGATTCCCCTGATGATGGATCAACTAGGTTCATACC
TTCACCCATGACTAGTCCCGGGTCCATTGATGAGTGCAGCTTCCCTGAGAGCCCTTA
G
SEQ ID NO: 4 sets out the DNA sequence of the MATA2
gene of a Yarrowia lipolytica strain
ATGGAAAACACGATACTACACATTCATTCCTTTCAACTACCCCAAACAGAACAGCCC
TACCCCGAGGCTATGTTATTCGACAGGGACACTAGCGATTCACGTACGGTTCTAACT
CAGAAGCCAAATGGACTGGAAATATCACTCAACTTTTTGCAATATGACGGTCACAA
GGGGCTGTTCATCAGGCAAGACAGTCGAACGAATGAGCCTGAGTACATTGAACCCA
AAGTTCTGAAACCTAGGAACAGCTACATCTTGTTCCGAAATGCAACATCCAGATGCT
CTCAGAGCATTGATCCCAACTCAGCATTTGTTTCGAGGGTCTCCAGTTATATCTGGG
GCTCGGGAATCCCCAATCCTATTGTTCGGCGATGGTTCAAGTATATGGGTTTCTTCG
AAGCCCTATACCACGAAGTCGACTATCCTGAGTATATCCCGAACAAACTGCCCAAGT
CCCCAAAGAAGCCGAAGGTCCAGAAGGGTGCAACTAAGTCTAAGAAAAAGGGAGC
AAAGGGTAAGAACAAGGTTACTGCTCTCCAAGTACTGGTCGGCCCTACCGTTGGTGT
CGGGGGAAGGATGACTGTTAGCCCAATGACTGCTTTCTTTAGCAACAACTCTCACGT
TACGTGCTATGACCCCAACTTTGTTCTCGATACACCTACTCGAGAATTCCTCATGATG
CCCCTGGATGATATCACTCTCCCATCGCAAGGACCAAGATCAGATTCACAGGAGCA
GCACGTCCCTCGGCAGCCTCCCGATGGGCAGGACTATTTTGACATTCTGGATATTGA
TGATTTCGTTTCTCCTTATGATGACTTGACTACTCGCTCGTTCACCAACAGGCAGCTT
TTTAAGCATGCCACACTGGGTTGA
SEQ ID NO: 5 sets out the DNA sequence of the MATB1
gene of a Yarrowia lipolytica strain
ATGAGGCAAGATGAGTATCATATAAAGCAAAGGATTTTTTCAATCTCAAAAGCAGC
TATAACAATGATCAAGATAAAAACCGAGCACAAAGTGGTGAAGGCAAGAACCCCG
AAAAAAATGAAACCTGGAATGGCCCGTTTCAGAATTCAAGCCGTTCCCATCATCAAC
CAAAACAACAACGCCTACCTGTCAGAGATCAGCTTCGAAGAAAAGTTAGAACCAGT
TCCGCCGATGTATCCTGAACTACAAAAAGTCCTTGATTACATTCACTCACGCAAAGT
CCCCTTCGACGGATCATCGGGGCCATACCCTCCCTCGAGCAGACGACAAGTGGTCA
ACGGTTATGTCGCATTTAAGCTATATCACCACGTTCCACACACTGACCTTAGCGCAA
TTGATATCACCCACCTCCTCCAAGGTCCTTGGAGAGCTTGCCCTGATAGACACATCT
GGACCAAGTACGCTGATCACTACCGTTCTAGAGGGCGTGACGTCGCATTCAAGACAT
GGCTTCTGTCTGTTACTAGTCCTGCTCCTGAGAAGGGGCTAATTATTCAGCCTCAAA
CTCAGAGCGAGCAGGGGTACAACTTCTCATCTTCCTCTGATTTTTCAAGTTCTCCAGA
ACACAGTGAAACACTCCCTGTGTCATCTGGAGAGCCCAGTACACCTGTTGACCCCTT
CCTGATCGAGTTTGATCAAGGCATCGCGGAGAAAGAGGCCAGCTTCACAAGTCCTT
GCAACCAAAACTATGACTTGGATTTCAGCCAGCTTCAGCCTCTAAGTATTCGCGTCC
CTTATTTTAACCAGTACGCTTCTTCCAGTAACTCTGGATTGGATGTTGACTACTTTGA
CGGTAGCACCCTAGACACTCCTAGCTACATCGACACTTTCATCACACCATTGCACTA
G
SEQ ID NO: 6 sets out the DNA sequence of the MATB2
gene of a Yarrowia lipolytica strain
ATGAAGGAAACCAGAGATCTTCTCTTCTACAACGAGAACTCCATCGACTTCAACTTC
TCTCCAGAAGAGATGGACACCCATCAAATGATGTGTGTCAACAATGACAGCCAACA
GGACACATCGTTCCTCACAGCTACTACTTGCATTTTTGACTTCCAGAAGGACATCTAT
GACGACCAAAACCTGATGTCCTTCGACCCCTTCTTTCCACAGCAGCGAGATATGGCT
TTGTGGATTGCTTCAGTGGGAAATGGGGCTATCGATTGGAGTACTAAACCTCAACTG
ACTCTTTCAAAGAAGATCCCTGACGTCGTCAACGAAAAAGGAGAGGAGATAACCCC
TGCTGACCTACAACGCCACTATGCCGATAAGCATGTCGCCAAACTTCCAGATCATTC
CTGCGATCTTTACTACCTTCTATTCAAGCATGCCAAATTCGAGGCTACCACCCTGAG
GATCCCATTGTCACTCCTCAATGACAACGGTACAGGAGCACTTGGCCCATTCGACGA
GATCAGATCGTTGACGGATGAAGAACAACGTCTTCTTACTCTGCTTAAGACAATTTC
ACATTACAGCTGGGTCACTAAACATGCGACCAAAGACTGTACGGGCCAACTGCTTG
ACCTGTGCAAAGAGGGCCTCCAGCTTGTGGAATCTATCCAGGTTAACAAAACTTTCA
ACGGTGTCATTCTGCATATCCTAGTCGCCTACTTTGGGAGAAGATCTACTGCTCTAA
GAGATGAGTTTCAACTGCACAAGCTCTTTCTTGAACAACGGCTTGACATTGAGTTGG
AAATCTTGGCCCAACATGGAGGCGCTGACGCCATTGATTCTGAAAAGCTGCGGAAG
GTCAAGAAACTGAGCGAACTGTGGGAGTGGTTCAATGCCATCCCGTACAACCACCA
CCCTAGCGTCTCTCAGATCGAGTTTATCGCTTCTCAGATCGGGGAGAAAGTAAGTTT
CGTGAAATCCTGGGTTGCAAACAAGCGACGAACTGGGGCCAAGAAGAGATCCAAGA
AACCGGCACCTTCGACACAGATTAGACCTGCATTGAAGGTATTCTTGGAGAAGAAG
CCATTTGTGTCTCGCCAAGTTGTGTAA

### DETAILED DESCRIPTION OF THE INVENTION

It is an objective of the invention to switch the mating type of a Yarrowia fungus industrial strain to an opposite type.

When a Yarrowia fungus strain is mutagenized, it produces a number of mutants, of which those with desired traits can be identified after screening. The process of mating type switch disclosed by this invention allows sexual crossing of such selected mutants and subsequently combines these advantageous genetic traits. By enabling mating type switch, a selected mutant can be further improved by taking up the advantageous genetic trait of another selected mutant of the same mating type.

Although mating type switch has been practiced before, it is the first time that this technique is successfully practiced in industrial Yarrowia fungus strains. An industrial Yarrowia fungus strain is a Yarrowia fungus strain which produces one or more product of interest, often of industrial use. In one embodiment, the making of product of interest industrial is caused by the genetic modification made to a Yarrowia fungus strain.

Furthermore, it is a new and surprising finding by the inventors of the present invention that by making mating type switch, the present invention allows quick and efficient combination of advantageous genetic traits of strain that are of the same mating type.

In an embodiment of the invention, the mating type of a Yarrowia fungus strain is switched to an opposite mating type by introducing one or more mating type locus gene of a Yarrowia fungus strain with an opposite mating type. The process begins with an acceptor Yarrowia fungus strain whose mating type is to be switched. This acceptor Yarrowia fungus strain has a mating type. Subsequently, one or more mating type locus gene of a Yarrowia fungus strain (the donor Yarrowia fungus strain) that is of the opposite mating type of the acceptor strain is introduced into the acceptor strain and thus causes the switch of the mating type of the acceptor Yarrowia fungus strain.

In one embodiment, a suitable acceptor Yarrowia fungus strain is a Yarrowia lipolytica strain. In a preferred embodiment, the suitable acceptor Yarrowia fungus strain is an industrial Yarrowia lipolytica strain. In a specific embodiment, the suitable acceptor Yarrowia fungus strain is Yarrowia lipolytica strain ML15186 or its derivative strains.

In one embodiment, the donor Yarrowia fungus strain is of the same species of the acceptor Yarrowia fungus strain. In another embodiment, the donor Yarrowia fungus strain is of another species from the same genus as the acceptor Yarrowia fungus strain.

The mating locus to be inserted into the acceptor strain must be of the opposite mating type of the acceptor strain. In one embodiment, if the acceptor Yarrowia fungus strain has a MAT-B mating type, the to-be-inserted mating locus may be a MAT-A mating locus. If the acceptor Yarrowia fungus strain has a MAT-A mating type, the to-be-inserted mating locus may be a MAT-B mating locus.

In one embodiment, the MAT-A locus comprises MATA1 gene and MATA2 gene. In another embodiment, the MAT-B locus comprises MATB1 gene and MATB2 gene.

The MAT-A locus according to embodiments herein may include, for example and without limitation, a polynucleotide comprising a nucleic acid sequence having at least at least 80%, at least 85%, at least 90%, at least 91%, at least 92%, at least 93%, at least 94%, at least 95%, at least 96%, at least 97%, at least 98%, or at least 99% identity to SEQ ID NO:1.

The MAT-B locus according to embodiments herein may include, for example and without limitation, a polynucleotide comprising a nucleic acid sequence having at least 80%, at least 85%, at least 90%, at least 91%, at least 92%, at least 93%, at least 94%, at least 95%, at least 96%, at least 97%, at least 98%, or at least 99% identity to SEQ ID NO:2.

The MATA1 locus according to embodiments herein may include, for example and without limitation, a polynucleotide comprising a nucleic acid sequence having at least 80%, at least 85%, at least 90%, at least 91%, at least 92%, at least 93%, at least 94%, at least 95%, at least 96%, at least 97%, at least 98%, or at least 99% identity to SEQ ID NO:3.

The MATA2 locus according to embodiments herein may include, for example and without limitation, a polynucleotide comprising a nucleic acid sequence having at least 80%, at least 85%, at least 90%, at least 91%, at least 92%, at least 93%, at least 94%, at least 95%, at least 96%, at least 97%, at least 98%, or at least 99% identity to SEQ ID NO:4.

The MATB1 locus according to embodiments herein may include, for example and without limitation, a polynucleotide comprising a nucleic acid sequence having at least 80%, at least 85%, at least 90%, at least 91%, at least 92%, at least 93%, at least 94%, at least 95%, at least 96%, at least 97%, at least 98%, or at least 99% identity to SEQ ID NO:5.

The MAB-2 locus according to embodiments herein may include, for example and without limitation, a polynucleotide comprising a nucleic acid sequence having at least 80%, at least 85%, at least 90%, at least 91%, at least 92%, at least 93%, at least 94%, at least 95%, at least 96%, at least 97%, at least 98%, or at least 99% identity to SEQ ID NO:6.

The introduction of opposite mating type locus gene(s) into the acceptor Yarrowia fungus strain is done by methods including but not limited to: insertion of an opposite type mating locus into the acceptor's mating locus, or partial or full replacement of the acceptor's mating locus with an opposite type mating locus.

Accordingly in an embodiment of the invention, the acceptor strain comprises the MAT-A locus into which the donor strain MAT-B locus is introduced. In one embodiment thereof, the MAT-A locus is replaced by a MAT-B locus. In another embodiment, a MAT-B locus is inserted into the MAT-A locus, resulting in an acceptor strain bearing the MAT-B mating type.

In another embodiment of the invention, the acceptor strain comprises no MAT locus, and from the donor strain MAT-A locus is introduced, resulting in a strain with MAT-A mating type. In another embodiment, the acceptor strain comprises no MAT locus, and from the donor strain a MAT-B locus is introduced, resulting in a strain with MAT-B mating type.

In the context of the present invention, the terms “recombination” and “recombinant” refers to any genetic modification not exclusively involving naturally occurring processes and/or genetic modifications induced by subjecting the host cell to random mutagenesis but also gene disruptions and/or deletions and/or specific mutagenesis, for example. Consequently, combinations of recombinant and naturally occurring processes and/or genetic modifications induced by subjecting the host cell to random mutagenesis are construed as being recombinant.

Recombination includes introduction and/or replacement of genes and may be executed by the skilled person using molecular biology techniques known to the skilled person (see Sambrook et al. or Ausubel et al. (J. Sambrook, E. F. Fritsch, T. Maniatis (eds). 1989. Molecular Cloning: A Laboratory Manual. Cold Spring Harbor Laboratory Press: New York; F. M. Ausubel, R. Brent, R. E. Kingston, D. D. Moore, J. G. Seidman, J. A. Smith, K. Struhl (eds.). 1998. Current Protocols in Molecular Biology. Wiley: New York)

It is another objective of the invention to provide a process to sexually cross different strains of Yarrowia fungus strains in which the chromosomal properties of the different strains can be combined in a new individual. In a preferred embodiment, such chromosomal properties are desired traits that are the results of mutagenesis of an ancestor strain. It is another objective of the invention to provide a process to pass on the above combined advantageous genetic traits into a progeny strain.

In an embodiment, the invention relates to a process for producing Yarrowia fungus strain progeny, wherein an acceptor Yarrowia fungus strain whose mating type is switched into an opposite mating type as defined above is crossed with a Yarrowia fungus strain which has the mating type of the original acceptor strain, and their progeny is isolated.

In one embodiment, a library of progeny of the crossed fungus strain described in the paragraph above is screened and one or more strains with a desired trait is selected. In one embodiment, the selected progeny has a trait that enhances production of a product of interest over any one of the two individual strains before they are crossed. In another embodiment, the selected progeny has a trait that reduces the level of production over any one of the two individual strains before they are crossed. In one embodiment, such trait that is a reduced level of production of an undesired product, such as a toxin.

The above invention helps to recombine properties of two strains of the same species in an effective way, i.e., by sexual crossing. The advantage of the current invention to the traditional method of parallel strain development of two opposite sex haploids is that it only requires the development of one line of strain and saves the time and effort of developing in parallel another line of strain of an opposite sex haploid for crossing purposes. When crossing is needed for the strain of the present invention, the mating type of the strain can simply be switched genetically to its opposite mating type in a simple recombinant maneuver. Further, because there is no parallel development of a strain of opposite mating type, there is no need to check the genetic makeup of the opposite sex haploid before mating as needed in the parallel strain development scheme.

A number of Yarrowia fungi strains, especially, Yarrowia lipolytica, have been used as host cells in the production of various compounds. A compound of interest may be any product that may be of industrial use. The compounds of interest of the present invention can be any fine chemical or biological compound. The terms “compound of interest” and “product of interest” are used interchangeably in this application.

The term “biological compounds” is known in the art and includes compounds which are the building blocks of an organism. Examples of biological compounds include, but are not restricted to: proteins, polypeptides, amino acids, nucleic acids, nucleotides, carbohydrates, and lipids.

The term “fine chemical” is known in the art and includes compounds which are produced by an organism and are used in various branches of industry such as, for example but not restricted to, the pharmaceutical industry, the agriculture, cosmetics, food and feed industries. These compounds include, for example, steviol glycoside, tartaric acid, itaconic acid and diaminopimelic acid, lipids, saturated and unsaturated fatty acids (e.g., arachidonic acid), diols (e.g. propanediol and butanediol), aromatic compounds (e.g., abieno, sclareol, beta-ionone, aromatic amines, vanillin and indigo), carotenoids, vitamins and cofactors.

Higher animals have lost the ability to synthesize vitamins, carotenoids, cofactors and nutraceuticals and therefore need to take them in, although they are easily synthesized by other organisms such as bacteria. These molecules are either biologically active molecules per se or precursors of biologically active substances which serve as electron carriers or intermediates in a number of metabolic pathways. These compounds have, besides their nutritional value, also a significant industrial value as coloring agents, antioxidants and catalysts or other processing aids. The term “vitamin” is known in the art and includes nutrients which are required by an organism for normal functioning, but cannot be synthesized by this organism itself. The group of vitamins may include cofactors and nutraceutical compounds. The term “cofactor” includes non-protein compounds which are necessary for the occurrence of normal enzymatic activity. These compounds may be organic or inorganic; the cofactor molecules of the invention are preferably organic. The term “nutraceutical” includes food additives which promote health in organisms and animals, especially in humans. Examples of such molecules are vitamins, antioxidants and likewise certain lipids (e.g., polyunsaturated fatty acids).

Preferred fine chemicals or biosynthetic products which can be produced in organisms of the genus Yarrowia are carotenoids such as, for example, phytoene, lycopene, beta-carotene, alpha-carotene, beta-cryptoxanthin, lutein, zeaxanthin, astaxanthin, canthaxanthin, echinenone, 3-hydroxyechinenone, 3′-hydroxyechinenone, adonirubin, violaxanthin and adonixanthin, and aromatic compounds such as abienol, sclareol, ionone, and sweeteners such as steviol glycoside, and many other compounds.

In the production methods according to the present invention, the Yarrowia fungus strains according to the invention are cultivated in a nutrient medium suitable for production of the compound of interest, e.g., fine chemicals or biological compounds, using methods known in the art. Examples of cultivation methods which are not construed to be limitations of the invention are submerged fermentation, surface fermentation on solid state and surface fermentation on liquid substrate. For example, the cell may be cultivated by shake flask cultivation, small-scale or large-scale fermentation (including continuous, batch, fed-batch, or solid state fermentations) in laboratory or industrial fermenters performed in a suitable medium and under conditions allowing efficient production of the compound of interest. The cultivation takes place in a suitable nutrient medium comprising carbon and nitrogen sources and inorganic salts, using procedures known in the art. Suitable media are available from commercial suppliers or may be prepared according to published compositions. If the fine chemicals or biological compounds are secreted into the nutrient medium, the fine chemicals or biological compounds can be recovered directly from the medium. If the fine chemicals or biological compounds are not secreted, it can be recovered from cell lysates.

The resulting compound of interest may be recovered by the methods known in the art. For example, the fine chemicals or biological compounds may be recovered from the nutrient medium by conventional procedures including, but not limited to, centrifugation, filtration, extraction, spray-drying, evaporation, or precipitation.

Polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulphate precipitation), SDS-PAGE, or extraction.

The invention described and claimed herein is not to be limited in scope by the specific embodiments herein enclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In case of conflict, the present disclosure including definitions will be taken as a guide.

The following examples are intended to illustrate the invention without limiting its scope in any way.

### EXAMPLES

### Example 1

Generating Mutant Strains of Yarrowia lipolytica

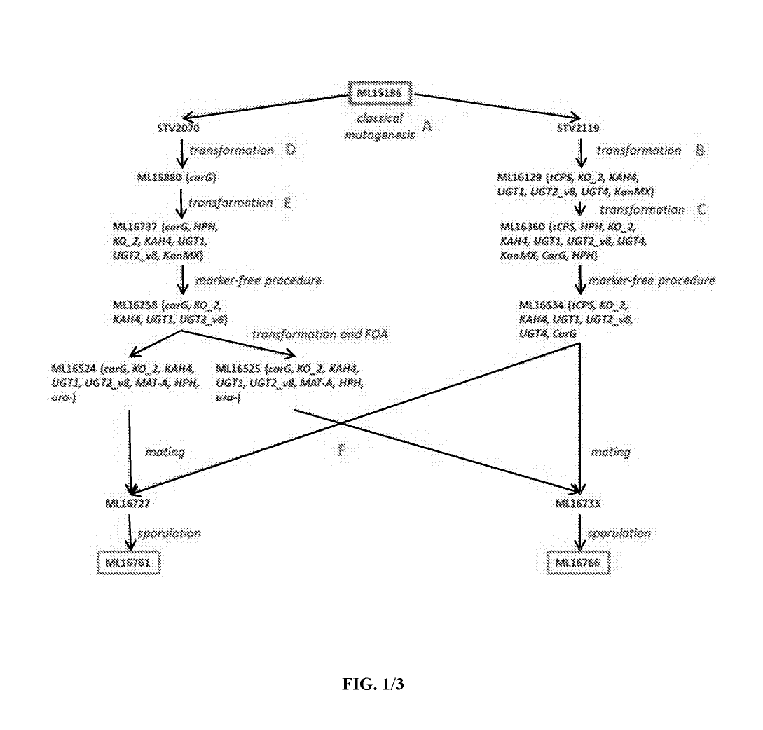

In this example, the Yarrowia lipolytica strain ML15186, which has the native MAT-B mating type locus, was mutagenized, leading to mutant strains STV2070 and STV2119 (FIG. 1, A).

Strain ML15186 was grown in 500 ml shake flasks containing 100 ml YEPD with 2% glucose at 280 rpm and 30° C. At an optical density (OD₆₀₀) of 2, cells were spun down and washed twice with milliQ water and suspended in 25 ml 0.1M sodium citrate buffer pH 5.5. To 4 ml of the cell suspension NTG was added to a final concentration of 0.150 mg/ml. The cell suspension was then incubated for 45 minutes in a water bath at 25° C. and 75 rpm. The reaction was stopped by addition of 1 ml 10% Na₂S₂O₃.5H₂O. The suspension was poured directly into 50 ml Greiner tubes, filled up to 50 ml with sterile 0.85% physiological salt solution, mixed and centrifuged. The pellets were washed once and re-suspended in 10 ml sterile physiological salt solution. Cells were plated for single colonies, and tested for the production of steviol glycosides. One of these colonies was named STV2070.

Another ML15186 strain was grown in 500 ml shake flasks containing 100 ml YEPD with 2% glucose at 280 rpm and 30° C. At an OD₆₀₀ of 2, cells were spun down and washed twice and re-suspended in milliQ water to a final OD of 1.0. 10 ml of this cell suspension was transferred to a plastic petri dish and exposed to UV using the DARK TOP UV (Osram HQV 125W) for 300 sec with gentle shaking at 6 rpm. After UV mutagenesis the suspension was kept in the dark for 2 hours to avoid photo reactivation. Cells were plated for single colonies, and tested for the production of steviol glycosides. One of these colonies was named STV2119.

### Example 2

Preparation of Strains with Improved Steviol Glycosides Producing Function from Strain Stv2119

In this experiment, strains with steviol glycosides producing activity were made by introduction of tCPS_SR, KAH_4, UGT4, UGT1 and UGT2_v8 in strain STV2119 (FIG. 1, B).

Strain STV2119 was transformed with two DNA fragments produced by PCR and purified following column purification. One fragment encodes part of the Y. lipolytica GSY1 gene, the tCPS_SR linked to the Y. lipolytica pSCP2 promoter and gpdT terminator, the KAH_4 linked to the synthetic Y. lipolytica pENO promoter and pgmT terminator and the pAgos_lox_TEF1p promoter with a lox site and part of the KanMX marker. This fragment was amplified with oligos GSY1-F and KAN-R.

The other fragment encodes for a complementary part of the KanMX marker with a Agos_teflTs_lox terminator also containing a lox site. In addition, the latter fragment encodes for UGT4 linked to the synthetic Y. lipolytica pHSP promoter and pgkT terminator, UGT1 linked to the synthetic Y. lipolytica pHYP promoter and act1T terminator, UGT2_v8 linked to the synthetic Y. lipolytica pYP005 promoter and pdc1T terminator, and part of the Y. lipolytica GSY1 gene. This fragment was amplified with KAN-F and GSY1-R. Both fragments contain part of the GSY1 gene for targeted integration at this locus, and assemble into one construct in Y. lipolytica upon transformation and genomic integration. See Appendix XIII for a schematic representation. After transformation cells were plated on YEPD with 400 μg/ml G418. A G418-resistant and RebA-producing colony was named ML16129.

### Example 3

Preparation of Strains with Increased Geranylgeranyl Pyrophasphate (GGPP) Production Activity from Strain ML16129

In this experiment, the ability of the Y. lipolytica cell to produce a terpenoid intermediate product, GGPP, was increased by the introduction of CarG gene in strain ML16129 (FIG. 1, C).

Strain ML16129 was transformed with a 4.4 kb fragment isolated by gel purification following PvuII digestion of plasmid MB7282. MB7282 encodes CarG linked to the native Y. lipolytica pHSP promoter and cwpT terminator and also encoding the HPH hygromycin resistance gene flanked by lox sites. Transformants were selected on YPD with 100 ug/ml hygromycin. A selected hygromycin resistant transformant was denoted ML16360.

### Example 4

Removal of Marker from Strain ML16360

In this experiment, HPH hygromycin resistance marker was removed from the host cell so that the same marker can be reused in later experiments (FIG. 1).

The HPH antibiotic marker was removed from strain ML16360 after transformation with MB6128 which encodes a construct for constitutive overexpression of the CRE recombinase. After selection of MB6128 transformants on YPD+G418 and screening for transformants that lost HYG resistance by successful Cre-Lox recombination, the sensitive colonies were grown on non-selective medium to remove the MB6128 CEN plasmid (spontaneous loss of the CEN plasmid). The resulting antibiotic marker-free variant was denoted ML16534.

### Example 5

Preparation of Strains with Increased Geranylgeranyl Pyrophasphate (GGPP) Production Activity from Strain STV2070

In this experiment, the ability of the Y. lipolytica cell to produce an terpenoid intermediate product, GGPP, was increased by the introduction of the carG gene in strain STV2070 (FIG. 1, D).

Strain STV2070 was transformed with a 4.2 kb fragment isolated by gel purification following PvuII digestion of plasmid MB7351. MB7351 encodes CarG linked to the native Y. lipolytica pTPI promoter and xprT terminator and also encodes the HPH hygromycin resistance gene flanked by lox sites. Transformants were selected on YPD with 100 ug/ml hygromycin. A selected hygromycin resistant transformant was denoted ML15880.

### Example 6

Preparation of Strains with Steviol Glycosides Producing Function from Strain ML15880

In this experiment, strains with steviol glycosides producing activity were made by introduction of KAH4, KO_2, UGT1 and UGT2_v8 in strain ML15880 (FIG. 1, E).

ML15880 was transformed with two DNA fragments produced by PCR and purified following column purification. One fragment encodes part of the Y. lipolytica GSY1 gene, the KO_2 linked to the Y. liplolytica pCWP promoter and pgkT terminator, the KAH_4 linked to the synthetic Y. lipolytica pHSP promoter and pgmT terminator and the pAgos_lox_TEF1ps promoter with a lox site and part of the KanMX marker. This fragment was amplified with oligos GSY1-F and KAN-R.

The other fragment encodes a complementary part of the KanMX marker with a Agos_teflTs_lox terminator also containing a lox site. In addition, the latter fragment encodes for UGT1 linked to the synthetic Y. lipolytica pHYP promoter and act1T terminator, UGT2_v8 linked to the synthetic Y. lipolytica pENO promoter and pdc1T terminator, and part of the Y. lipolytica GSY1 gene. This fragment was amplified with KAN-F and GSY1-R. Both fragments contain part of the GSY1 gene for targeted integration at this locus, and assemble into one construct in Y. lipolytica upon transformation and genomic integration. After transformation cells were plated on YEPD with 400 μg/ml G418. A G418-resistant and RebA-producing colony was named ML16137.

### Example 7

Removal of Marker from Strain ML16137

In this experiment, the HPH hygromycin resistance marker was removed from the host cell so that the same marker can be reused in later experiments (FIG. 1).

The HPH and KAN antibiotic markers were removed from strain ML16137 after transformation with MB6129, which encodes a construct for constitutive overexpression of the CRE recombinase. After selection of MB6129 transformants on YPD+nourseothricin and screening for transformants that lost HYG and G418 resistances by successful Cre-Lox recombination, the sensitive colonies were grown on non-selective medium to remove the MB6129 CEN plasmid (spontaneous loss of the CEN plasmid). The resulting antibiotic marker-free variants was denoted ML16258.

### Example 8

### Mating Type Switch of Strain ML16258

In this example, the mating type of ML16258 is switched from MAT-B to MAT-A (FIG. 1).

Strain ML16258 (MAT-B) was transformed with a 6.1 kb fragment isolated by gel purification following BbsI digestion of plasmid pMB7293. pMB7293 encodes 1491 bp 5′ to the native Y. lipolytica MAT-A locus, the HPH hygromycin resistance gene flanked by lox71/lox66 sites, the native Y. lipolytica MATA2 and MATA1 genes, and 2209 bp 3′ to the native Y. lipolytica MAT-A locus. The flanking 5′ and 3′ flanking regions each contain a BbsI site such that the fragment isolated following BbsI digestion contains ˜1 kb of of the flanking sequence allowing for homologous recombination into the MAT locus.

Transformants were selected on YPD with 100 ug/ml hygromycin. PCR was used to screen for integration of the construct at the MAT locus and a selected MAT-A, hygromycin resistant transformant was denoted ML16523.

### Example 9

Mating of MAT-A Strain with MAT-B Strain

In this experiment, MAT-A strain ML16523 is mated with MAT-B strain ML16525 and with MAT-B strain ML16534, and the resultant diploids were sporulated (FIG. 1, F).

Strain ML16523 was streaked to YPD and grown overnight and then streaked to 5-FOA plates to allow for recombination mediated loss of the URA2 marker. Two selected 5-FOA resistant transformants were denoted ML16524 and ML16525.

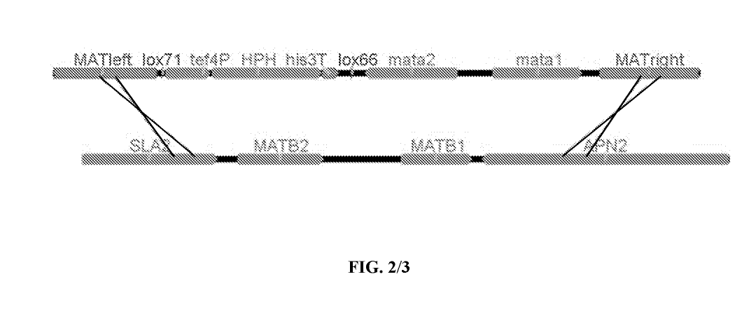

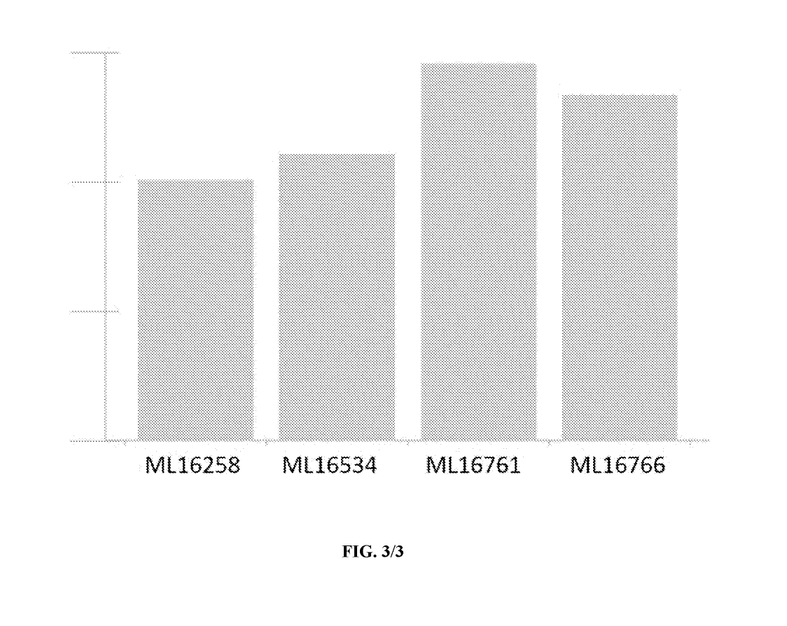

Strains of opposite mating types (ML16524 and ML16534 or ML16525 and ML16534) with complementary nutritional deficiencies and antibiotic sensitivities (URA2+ hyg- and ura2-HYG+) were allowed to mate and then plated on selective media that would allow only diploids to grow (minimal media with 100 ug/mL hygromycin). Diploid cells (ML16727 and ML16733, respectively) were then induced to undergo meiosis and sporulation by starvation, and the resulting haploid progeny colonies were replica-plated to identify prototrophic isolates with hygromycin sensitivity (FIG. 2). Selected rebaudioside A-producing strains were denoted ML16761 (from parent ML16727) and ML16766 (from parent ML16733) (FIG. 3).

### Example 10

### Mating Type Switching of Carotene Producing Strain for Increased Carotene Titer in Genetic Crossed Progeny

Strain ML5252 (MATA) is converted from MAT-A to MAT-B as shown in Example 8, but with plasmid pMB7294 cut with SfiI to release a 6.8 kb DNA fragment containing lox flanked hygromycin resistance and MAT flanking regions. Specifically, plasmid pMB7293 encodes 1491 bp 5′ to the native Y. lipolytica MAT-B locus, the HPH hygromycin resistance gene flanked by lox71/lox66 sites, the native Y. lipolytica MATB2 and MATB1 genes, and 2209 bp 3′ to the native Y. lipolytica MAT-A locus.

Transformants are selected on YPD medium with 100 ug/ml hygromycin. PCR is used to screen for integration of the construct at the MAT locus. A selected MAT-B, hygromycin resistant transformant is denoted MLcaro-mat strain.

This MLcaro-mat strain is subsequently submitted to mutagenesis, as in Example 1 and genetic modification by transformation of mevalonate pathway and carotenoid pathway genes such as, but not limited to geranylgeranyl pyrophosphate synthase (GGPPS) as in Example 5. These strains are mated to the progenitor strain ML5252, and made to sporulate as in Example 9. The resulting haploid isolates are examined for increased titer for carotene.

### Example 11

### Mating Type Switching of Ionone Producing Strain for Increased Ionone Titer in Genetic Crossed Progeny

The β-ionone-producing Yarrowia strain ML15449 is constructed from strain ML5252 by the deletion of Yarrowia ALK1 and ALK2 genes, followed by introduction of 3 copies of Yarrowia codon optimized the Petunia CCD1 gene. The Petunia CCD1 gene is driven by the TEF1 promoter.

Strain ML15449 (MATA) was converted from MAT-A to MAT-B by transformation with pMB7294 and screening as in Example 10. A selected MAT-B, hygromycin resistant transformant is denoted MLionone-mat_strain

This MLionone-mat strain is subsequently submitted to mutagenesis, as described in Example 1 and genetic modification by transformation of mevalonate pathway and carotenoid pathway genes such as, but not limited to geranylgeranyl pyrophosphate synthase (GGPPS) as in Example 5. These strains are mated to progenitor strain, ML15449, and made to sporulate as in Example 9. The resulting haploid isolates are examined for increased titer for ionone.

1. A process for switching the mating type of a Yarrowia fungus strain to an opposite mating type, wherein an acceptor Yarrowia fungus strain is subject to genetic modification in which one or more mating type locus genes (MAT) of the opposite mating type of the acceptor Yarrowia fungus strain is introduced into the acceptor Yarrowia fungus strain and thus switches the acceptor Yarrowia fungus strain to the opposite mating type.

2. The process of claim 1, wherein the acceptor Yarrowia fungus strain is Yarrowia lipolytica.

3. The process of claim 1, wherein the acceptor Yarrowia fungus strain is an industrial strain.

4. The process of claim 1, wherein the acceptor Yarrowia fungus strain has a MAT-B locus, and in which a MAT-A locus is introduced.

5. The process of claim 4, wherein the MAT-A locus consists of a MATA1 gene and a MATA2 gene.

6. The process of claim 1, wherein the acceptor Yarrowia fungus strain has a MAT-A locus, and in which a MAT-B locus is introduced.

7. The process of claim 6, wherein the MAT-B locus consists of a MATB1 gene and a MATB2 gene.

8. The Yarrowia fungus strain obtained by the process of claim 1.

9. The Yarrowia fungus strain of claim 8, wherein said Yarrowia fungus strain produces one or more product of interest.

10. The Yarrowia fungus strain of claim 9, wherein said one or more product of interest is steviol glycoside.

11. The Yarrowia fungus strain of claim 9, wherein said one or more product of interest is carotenoid or beta-ionone.

12. A process for producing Yarrowia fungus strain progeny for industrial production, wherein parent Yarrowia fungus strains with two opposite mating types are sexually crossed and their progeny is isolated, and wherein one of the parent strains is generated according to the process of claim 1.

13. A process for selecting a Yarrowia fungus strain with a desired phenotype, wherein a library of progeny produced in accordance with claim 12 is screened, and one or more strains with a desired phenotype is selected.

14. A Yarrowia fungus strain obtainable by the process of claim 12, wherein said Yarrowia fungus strain produces one or more product of interest.

15. The Yarrowia fungus strain of claim 14, wherein said one or more product of interest is steviol glycoside.

16. The Yarrowia fungus strain of claim 14, wherein said one or more product of interest is carotenoid or beta-ionone.

17. A process for the preparation of one or more compound of interest, comprising:
a. cultivating a Yarrowia fungus strain according to claim 14 under conditions conducive to the production of said compound; and
b. recovering said compound of interest from the cultivation medium or cell lysates.


SEQUENCE LISTING (6 sequences)

SEQ ID NO: 1 - DNA, 5995 bp, Yarrowia lipolytica
  misc_feature (1453)..(1455): n is a, c, g, or t
caacaggccatggaggaggagcgcatccggcagatgcagatgcagcagcagcagcagtttgaaatgcagcagcgacagcagatggaagcgcaacagcgggctcaggaacaactaatggccgaccagatggcccgacatgcccagggtcgaatggccgagcttgagcgagacattctggctctccgaggtcagtacgatcaggatcagctcatgctggagcagtacgatggtcgagtaaaggctctggaggctgagctgaatcagctgcagcagactgctcatcagagygcccaggccaaggatgatctgattgaatctcttcagcagcagatcaccatgtggcggcaaaagtacgagactctggccaaacggtactcgtccatgcgagaggaatatcttgccttgctcaagaagctcaaggctacccaacagaaggctgcgtcagctaaggaagccattgagaaggctgagaagmtggagcgagacatgcgacacaagaacattgagcttgctgatctcatcaaggagcgagatcgagctcgatatgatcttgaccgtgccaagggtggcaacaaggaggacgtggagcgtctggaacgagagcttcgaatggcccaggacaagctggccgataaggaccgatctaccggtgctgatctgtctcttcttttgtcgaagcataaccgagagctttcagagcttgagaatgctttgaagatgaagcagcgggctctggacgagcgaggagatgactctgatctgttgagacgactggaagagaaggagattgagtacgaggctctcaacgaagccttcaactcgctggctctgcaccaggagcagcagggcaacaacagcaacggtggatccactcccctggctgctttgcattctatcattgatgctcttctagagtccggttcccagcgtgttcaggacgctctcttcgagctcgagtcgcctatgcaggccggtaaccagaactcgactcccgagtatctcttgtctgtcatcgagaaggcgtccgagtctgcttccgcctttgccacgtcgttcaacaacttcttggccgacgaaaccgatggcgactacgccgagatcatcaagaccgtcaacatctactctactgctgtggagaatgttctgtccaactccaagggtctcactcgtctggccaaggacgatgcttctgccgacgcccttgtcaactttgctcgtgagtctgccgaggccacagagcgaaactttattggtcttctgtctgagatcattgaggatttccctctggatgacaagatggagcgggtcatcactctcaacatgaacgtgcagactgctcttcagcacctcaccaagctggccgagcgaatggcccccaagaccaacatcaacatgtctggtgatctgggcgacctcgttgagcgagagatggccaaggctgctgatgccattnnngctgctgcttctgctaagcttggcgatctgctcaagtttaaccagtccgaccctctcaagtcgaccaccgacctgcagctgcacgaggctgccatccaggctgcccaggccgtcatcaacgccattgctgcgctgattcgagctgccaccgatgcccagaacgagattgtagcccagggacgaggtacttcttctcgagcacagttctacaagaagaacaacaagtggaccgagggtcttatttccgcggccaagagtgtagctgcatcgaccaacattctcatcgagaaggccgacggcactctccgacgaacctctggtctcgaggagctcattgtggcttccaacaacgtcgctgcttccactgctcagctggttgctgcctctcgagtaaaggctaccttcatgtccaagacccaggacaagctggaagagtgttcaaaggtggttacttccgcctgccgaaacctcgtcaagcaggtgcaggaaattctcaacaagaagtttggcgagctggacgaaaaggtagactacgctgccctctccaagcacgagttcaagacgaccgaaatggaacagcaggtcgagattctcaagctcgaaaacgatctgcagggcgcccgaaagcgacttggacagatgcgaaaggtcgcctacctgcatcaggacgaggaggagtccattcctggccaggaggactaagctacgcgcggctgatgtatgtataacatgtatggtaaatgaatgaaatgatatttttagtgatcgaatgatgaagaaggctctgcgatgctttactcgttacatgacaactgtccaatgcgacagttctatgttactatgacacacactatttattacgatgcaaattatgttgacatgcatgagacaaaccccactgtaaccactttattagaaatgctaagggctctcagggaagctgcactcatcaatggacccgggactagtcatgggtgaaggtatgaacctagttgatccatcatcaggggaatccgtcttgggctctctgaaccgacgattggtgaaccattgtgtaacctgatgaagtgagattccacaatgctctccaataattctcctctctgttctggttggatgagaacagatcttgaacagagagtcaagataggcggtaacctcgttacttaacctattccggactttcttaggaaagacgtcacagttgtgatccagactctgcaacttgtcagcttcttttagagctctggctttgacttcgaggtctttggtgctggcctcataggctctgatttcgcctctatgtctttgtctaaatttgctgcgacctctagatagtcatcaagcagctctgcggcaattccttttgcaagactgtactcatcattccaatcagcacccagtatggccttactggtcatccattcgttctgttttagtattccccaatactgctcttgttttcaatatactcactaatctcgctggtaccagttgctggatgtaagtgatgtagtggcgaatcagcctgtcgtatttgattctgtgttcatttggaacaccctccaaaaaggtccaaatcgttgtgcttatgtagagtccactctcagggaatttggacatccactcagaaatctcttcaaaccatttaagggaactggagatttcgattgactgttcgtgaagggcctggcacgtggtttcgccttttcttgcggccaagatcaggcgctggcagcgccagatatctgtaggggttcggctaggcatgtgatgtggggagaaaaaaaatgtcgtatctgtttaattaagtaccagggtgtacttggtggggccggtttctagatatatatttacaatggtcacttggactggtcagacgaggtggatgtggctagtttaccccgatattattcatgcacgccgtgcatatggttctggaacaaagacaccttaagcgacattggctcttgcctcatcacaattgcctaaactagagatctagtatttccagaccaacacatatcgacaaatatatataaacaacatgcaactctcttggctggctattatcagctataccaagccacacataacatcctcaaaatggaaaacacgatactacacattcattcctttcaactaccccaaacagaacagccctaccccgaggctatgttattcgacagggacactagcgattcacgtacggttctaactcagaagccaaatggactggaaatatcactcaactttttgcaatatgacggtcacaaggggctgttcatcaggcaagacagtcgaacgaatgagcctgagtacattgaacccaaagttctgaaacctaggaacagctacatcttgttccgaaatgcaacatccagatgctctcagagcattgatcccaactcagcatttgtttcgagggtctccagttatatctggggctcgggaatccccaatcctattgttcggcgatggttcaagtatatgggtttcttcgaagccctataccacgaagtcgactatcctgagtatatcccgaacaaactgcccaagtccccaaagaagccgaaggtccagaagggtgcaactaagtctaagaaaaagggagcaaagggtaagaacaaggttactgctctccaagtactggtcggccctaccgttggtgtcgggggaaggatgactgttagcccaatgactgctttctttagcaacaactctcacgttacgtgctatgaccccaactttgttctcgatacacctactcgagaattcctcatgatgcccctggatgatatcactctcccatcgcaaggaccaagatcagattcacaggagcagcacgtccctcggcagcctcccgatgggcaggactattttgacattctggatattgatgatttcgtttctccttatgatgacttgactactcgctcgttcaccaacaggcagctttttaagcatgccacactgggttgagcttggtaattttctgtactcactgtacgtttatcattggacacactaacggtattataatttaattttatttccactgaaagaagttacatctggcactggtgtcgtcctgggagcccttggcgtcggggtctgccgatgccttgtaggagtcgccgacgagctgcagcgagtggttctggccacaaacccaaaagtgcttgccctggttgggtccctttttgcgggtggttcttagcaccgccgggaggttgtgggcacagacgggggctggtttttttgtcatgatcttggaccactgcgacgcgaccttttcggcctcctccacgcgctccgcaatgtctaggtactcggtgggctctggcaagtccagtctggtggccatttgcggtctcgtatcttcaccctctcgagtcgcatgcgcacctgcatcgcaaccttgataggtgctttcaatatttggggacacggcgggtgattgtgattgtcccaacggctttgtgtgcacatccgagacgcatgttgatacgacggcagcttctggatctggcttttggaagaaggaggagatggccgcctgttttttggcagtcgatttggagggcgctttgggtttggtaatgcccttggtcggcggcgtttgcgcccggaacatggacatgaccgaggtggtctgcacaaactgcttgttagttctgtggaacagttttggccgaaacgagtttggggctgaggacgacgcttgtttggtctggttagctgcaccttgagtggcacggttgtctttttcttggcacgcgtctggttcgcgcacctctgcataaacgggacaatggtctgagccctccaggtggggcagaatatcggcatcctccaccttcagaccttttgaggccagcacgtagtcgatacgagacccaaagtttccgggccggtagttcatcatcacgttccagcaagtatacatgtcgggtcgattgggatgcttgttgcggcacgagtcctgtaacaggtcggggaccagtctgtggaagactcgtcggccaactttggcttccctccagctcttacactgtcccgggttgagcttttcaaacacctccacaaacctgccctcctccttatctcccagtgtcggtagagtgatgtgtttggccttgtgcagcgcttgcattccctcagctgagtcgtacagctcgcgtgccacattgagatctcccatgaccaccacctctcgtccagcctcaaccagaaggttgacccgttcctccagcaacccaaaatagtcgtctctgtaggcatctcgctcctcgccctcggtggcggtgctaggacagtagagcccaaacacgacacagaaccccaggtccaacacgagagaccggccctccgaatcgacctgaaagagccgctgtggattgccctcgggataggcgccgatacaactcttgctgctgccgtctctctccctttgccgctcgtacagctgctggtaggtgagtccctttttggagtccggagataccagctgaccggtaagaccctcctcggccttgag

SEQ ID NO: 2 - DNA, 8318 bp, Yarrowia lipolytica
atgagattcgtcacgttcaacatctgtggcatcaacaacgtgctgagctaccatccatggaacgagcaaaggtcgtttgagcacatgtttgcggtgctgaaggcggacgtgatctgtttacaagaaaccaagctccagccccatttgctgaagcgagaacacgcccttgtgcccggatacgattcgtactggtctttttccaacacaaaaaagggctacagcggcgtggcggtctatgtgaaacatggcatcaaagttctcaaggccgaggagggtcttaccggtcagctggtatctccggactccaaaaagggactcacctaccagcagctgtacgagcggcaaagggagagagacggcagcagcaagagttgtatcggcgcctatcccgagggcaatccacagcggctctttcaggtcgattcggagggccggtctctcgtgttggacctggggttctgtgtcgtgtttgggctctactgtcctagcaccgccaccgagggcgaggagcgagatgcctacagagacgactattttgggttgctggaggaacgggtcaaccttctggttgaggctggacgagaggtggtggtcatgggagatctcaatgtggcacgcgagctgtacgactcagctgagggaatgcaagcgctgcacaaggccaaacacatcactctaccgacactgggagataaggaggagggcaggtttgtggaggtgtttgaaaagctcaacccgggacagtgtaagagctggagggaagccaaagttggccgacgagtcttccacagactggtccccgacctgttacaggactcgtgccgcaacaagcatcccaatcgacccgacatgtatacttgctggaacgtgatgatgaactaccggcccggaaactttgggtctcgtatcgactacgtgctggcctcaaaaggtctgaaggtggaggatgccgatattctgccccacctggagggctcagaccattgtcccgtttatgcagaggtgcgcgaaccagacgcgtgccaagaaaaagacaaccgtgccactcaaggtgcagctaaccagaccaaacaagcgtcgtcctcagccccaaactcgtttcggccaaaactgttccacagaactaacaagcagtttgtgcagaccacctcggtcatgtccatgttccgggcgcaaacgccgccgaccaagggcattaccaaacccaaagcgccctccaaatcgactgccaaaaaacaggcggccatctcctccttcttccaaaagccagatccagaagctgccgtcgtatcaacatgcgtctcggatgtgcacacaaagccgttgggacaatcacaatcacccgccgtgtccccaaatattgaaagcacctatcaaggttgcgatgcaggtgcgcatgcgactcgagagggtgaagatacgagaccgcaaatggccaccagactggacttgccagagcccaccgagtacctagacattgcggagcgcgtggaggaggccgaaaaggtcgcgtcgcagtggtccaagatcatgacaaaaaaaccagcccccgtctgtgcccacaacctcccggcggtgctaagaaccacccgcaaaaagggacccaaccagggcaagcacttttgggtttgtggccagaaccactcgctgcagctcgtcggcgactcctacaaggcatcggcagaccccgacgccaagggctcccaggacgacaccagtgccagatgtaacttctttcaatggaaatgatcgaaggttggctattgtaacaaggaaactgccatttcagatgtttacaactgcttttgatatattatacttttataaaagatgaggatcgacaacgataatgcacaatgagcctgtttcaccaacactgtcatcccatttgggttaagtcaatatcagcgttcctattacctaaaaccgaagggtctactacttatatagtattggtagtttctttgacacgtccactctcgtcttacatatcataccactaccgaatgaaggaaaccagagatcttctcttctacaacgagaactccatcgacttcaacttctctccagaagagatggacacccatcaaatgatgtgtgtcaacaatgacagccaacaggacacatcgttcctcacagctactacttgcatttttgacttccagaaggacatctatgacgaccaaaacctgatgtccttcgaccccttctttccacagcagcgagatatggctttgtggattgcttcagtgggaaatggggctatcgattggagtactaaacctcaactgactctttcaaagaagatccctgacgtcgtcaacgaaaaaggagaggagataacccctgctgacctacaacgccactatgccgataagcatgtcgccaaacttccagatcattcctgcgatctttactaccttctattcaagcatgccaaattcgaggctaccaccctgaggatcccattgtcactcctcaatgacaacggtacaggagcacttggcccattcgacgagatcagatcgttgacggatgaagaacaacgtcttcttactctgcttaagacaatttcacattacagctgggtcactaaacatgcgaccaaagactgtacgggccaactgcttgacctgtgcaaagagggcctccagcttgtggaatctatccaggttaacaaaactttcaacggtgtcattctgcatatcctagtcgcctactttgggagaagatctactgctctaagagatgagtttcaactgcacaagctctttcttgaacaacggcttgacattgagttggaaatcttggcccaacatggaggcgctgacgccattgattctgaaaagctgcggaaggtcaagaaactgagcgaactgtgggagtggttcaatgccatcccgtacaaccaccaccctagcgtctctcagatcgagtttatcgcttctcagatcggggagaaagtaagtttcgtgaaatcctgggttgcaaacaagcgacgaactggggccaagaagagatccaagaaaccggcaccttcgacacagattagacctgcattgaaggtattcttggagaagaagccatttgtgtctcgccaagttgtgtaattgaatcataatgtattacaatgttgttaggtacatactgtagattgttttatatgcaatattgtgcccaatacacaaggtacagtatgtacaatacaatacctaatgttcatactgaacaccttatatacaagtacattaaggtagttgtatgtatgtacatactgtagtcactacagacgctatctttccagtgtgccgaccggtaactgaaagcaaatatagaagtacggtttaagaaagatatcatttaatcaaaggtgcgatatctagaggcttgccccattctttgtttgtgcaatgaacagggaacatgctatgcagtaacaccaaataatgtacttagtctaatcaccttctgttatatttagttatcttcgctcctcgtcttattgtcgtcaatctaagtaggtacggtcgccgcaaaaggaaatctcttgccgccgtcgctacagtgatcaagtagctgagctacgaaggtaattaaaatatttgttcgctttgctcatacgtcgttttaatcttaaatttcatctgattacacttcggcgaggagttgtttctggcatctcgcccttcgaccttgcttatgatttctgtgaccagaaacctaaattacccacaatgtgagttgcaaatgcaggcagctaattacagtacagtacagtacagtactgaactgtatgtacagtaggtcacgaagaagttaatacttgtgaggcagagtaccacttcacagggatgcaattgcaatgtagaaaaatactgtgaaagtaaagtattaaacctatccaagcaaaataaaagtgttgactcaaataattcaaccctttcctaccagcaagcaatggagaagtaaagacgagggactgccacccatcctcaagctgttactcaaggtttgaaaaagagttcggacactcaacttccccagccaaaatcgcataaacaaagaatctcgcgagattgggtttatcacctaatgaggtaatcctcacgatgaggcaagatgagtatcatataaagcaaaggattttttcaatctcaaaagcagctataacaatgatcaagataaaaaccgagcacaaagtggtgaaggcaagaaccccgaaaaaaatgaaacctggaatggcccgtttcagaattcaagccgttcccatcatcaaccaaaacaacaacgcctacctgtcagagatcagcttcgaagaaaagttagaaccagttccgccgatgtatcctgaactacaaaaagtccttgattacattcactcacgcaaagtccccttcgacggatcatcggggccataccctccctcgagcagacgacaagtggtcaacggttatgtcgcatttaagctatatcaccacgttccacacactgaccttagcgcaattgatatcacccacctcctccaaggtccttggagagcttgccctgatagacacatctggaccaagtacgctgatcactaccgttctagagggcgtgacgtcgcattcaagacatggcttctgtctgttactagtcctgctcctgagaaggggctaattattcagcctcaaactcagagcgagcaggggtacaacttctcatcttcctctgatttttcaagttctccagaacacagtgaaacactccctgtgtcatctggagagcccagtacacctgttgaccccttcctgatcgagtttgatcaaggcatcgcggagaaagaggccagcttcacaagtccttgcaaccaaaactatgacttggatttcagccagcttcagcctctaagtattcgcgtcccttattttaaccagtacgcttcttccagtaactctggattggatgttgactactttgacggtagcaccctagacactcctagctacatcgacactttcatcacaccattgcactagtcataatggtgtagacatgggtttgtacgagtactacgatacgacctcaaactccaacggtagtcctatttattcacatcgttaatatcatttcattcatttaccatacatgttatacatacatcagccgcgcgtagcttagtcctcctggccaggaatggactcctcctcgtcctgatgcaggtaggcgacctttcgcatctgtccaagtcgctttcgggcgccctgcagatcgttttcgagcttgagaatttcgacctgctgttccatttcggtcgtcttgaactcgtgcttggagagggcagcgtagtctaccttttcgtccagctcgccaaacttcttgttgagaatttcctgcacctgcttgacgaggtttcggcaggcggaagtaaccacctttgaacactcttccagcttgtcctgggtcttggacatgaaggtagcctttactcgagaggcagcaaccagctgagcagtggaagcagcgacgttgttggaagccacaatgagctcctcgagaccagaggttcgtcggagagtgccgtcggccttctcgatgagaatgttggtcgatgcagctacactcttggccgcggaaataagaccctcggtccatttgttgttcttcttgtagaactgtgctcgagaagaagtacctcgtccctgggctacaatctcgttctgggcatcggtggcagctcgaatcagcgcagcaatggcgttgatgacggcctgggcagcctggatggcagcctcgtgcagctgcaggtcggtggtcgacttgagagggtcggactggttaaacttgagcagatcgccaagcttagcagaagcagcagcaatggcatcagcagccttggccatctctcgctcaacgaggtcgcccagatcaccagacatgttgatgttggtcttgggggccattcgctcggccagcttggtgaggtgctgaagagcagtctgcacgttcatgttgagagtgatgacccgctccatcttgtcatccagagggaaatcctcaatgatctcagacagaagaccaataaagtttcgctctgtggcctcggcagactcacgagcaaagttgacaagggcgtcggcagaagcatcgtccttggccagacgagtgagacccttggagttggacagaacattctccacagcagtagagtagatgttgacggtcttgatgatctcggcgtagtcgccatcggtttcgtcggccaagaagttgttgaacgacgtggcaaaggcggaagcagactcggacgccttctcgatgacagacaagagatactcgggagtcgagttctggttaccggcctgcataggcgactcgagctcgaagagagcgtcctgaacacgctgggaaccggactctagaagagcatcaatgatagaatgcaaagcagccaggggagtggatccaccgttgctgttgttgccctgctgctcctggtgcagagccagcgagttgaaggcttcgttgagagcctcgtactcaatctccttctcttccagtcgtctcaacagatcagagtcatctcctcgctcgtccagagcccgctgcttcatcttcaaagcattctcaagctctgaaagctctcggttatgcttcgacaaaagaagagacagatcagcaccggtagatcggtccttatcggccagcttgtcctgggccattcgaagctctcgttccagacgctccacgtcctccttgttgccacccttggcacggtcaagatcatatcgagctcgatctcgctccttgatgagatcagcaagctcaatgttcttgtgtcgcatgtctcgctccagcttctcagccttctcaatggcttccttagctgacgcagccttctgttgggtagccttgagcttcttgagcaaggcaagatattcctctcgcatggacgagtaccgtttggccagagtctcgtacttttgccgccacatggtgatctgctgctgaagagattcaatcagatcatccttggcctgggcgctctgatgagcagtctgctgcagctgattcagctcagcctccagagcctttactcgaccatcgtactgctccagcatgagctgatcctgatcgtactgacctcggagagccagaatgtctcgctcaagctcggccattcgaccctgggcatgtcgggccatctggtcggccattagttgttcctgagcccgctgttgcgcttccatctgctgtcgctgctgcatttcaaactgctgctgctgctgcatctgcatctgccggatgcgctcctcctccatggcctgttgctgccgtagccgctcctgctcggcgtcgtactgttgctgggcgagcagcgcgtcctgggaccaaaagttctcaatcggctgagcctcagcaggaatagagggcgtaggagtcgccacaggcaagggagcgggctctggagacacagatcgggtctcggccacggacttgggtcgctgggggagtccaggtccgtcctcctcttcaagcaggttgggagggttcttgccgagctgcgggatagtcacaatggatcgcaggtacttgagagtagagcaatcgtagtaaaagtctctcagtcggtcgtgctgcgagtagaatcgctgccggagagactcaagagcctcgtcgttaccagtagacacatgcatggctcgcagcatggagatgacgaaccggtagattccgtacgactcggcaatcatgggaaccagagacgagattttgcactcgttggatcgggatcgctgcagcgacgagaagacaagtcgctggaagtcgtccactccgtcctggagggtcatcaggttcataatggcctcgtatccctcgttggggtcgttaacggttcgcagagaaatgtaatcctcgtactcaaacattccgttgaaggccgggtggtacttgtggaaggtgagcttctgcatgaggaaccggacatactcggagatgagcttgccataaccgtggttgccctggttaggaccagtgtagttgtttcctcgagccaacgactggatgaagttgacattggcctgggcatctctcagcgccgatgggtggccctcctgaagcaccttgtggatggtgatcagggccttgaagaccatgacatcgtcggtctgcaggggctgaattcgaatgccgttccagaaggccttggacgaccggtggtcatgggtgtacacaatgcatgctcgcacgtgtttccgcttgggcgcagactcgtctgtgttggtcgcctttttgatgttgacgttgaggtccacttcggctctgttggaagacat

SEQ ID NO: 3 - DNA, 850 bp, Yarrowia lipolytica
atgcctagccgaacccctacagatatctggcgctgccagcgcctgatcttggccgcaagaaaaggcgaaaccacgtgccaggcccttcacgaacagtcaatcgaaatctccagttcccttaaatggtttgaagagatttctgagtggatgtccaaattccctgagagtggactctacataagcacaacgatttggacctttttggagggtgttccaaatgaacacagaatcaaatacgacaggctgattcgccactacatcacttacatccagcaactggtaccagcgagattagtgagtatattgaaaacaagagcagtattggggaatactaaaacagaacgaatggatgaccagtaaggccatactgggtgctgattggaatgatgagtacagtcttgcaaaaggaattgccgcagagctgcttgatgactatctagaggtcgcagcaaatttagacaaagacatagaggcgaaatcagaggcctatgaggccagcaccaaagacctcgaagtcaaagccagagctctaaaagaagctgacaagttgcagagtctggatcacaactgtgacgtctttcctaagaaagtccggaataggttaagtaacgaggttaccgcctatcttgactctctgttcaagatctgttctcatccaaccagaacagagaggagaattattggagagcattgtggaatctcacttcatcaggttacacaatggttcaccaatcgtcggttcagagagcccaagacggattcccctgatgatggatcaactaggttcataccttcacccatgactagtcccgggtccattgatgagtgcagcttccctgagagcccttag

SEQ ID NO: 4 - DNA, 876 bp, Yarrowia lipolytica
atggaaaacacgatactacacattcattcctttcaactaccccaaacagaacagccctaccccgaggctatgttattcgacagggacactagcgattcacgtacggttctaactcagaagccaaatggactggaaatatcactcaactttttgcaatatgacggtcacaaggggctgttcatcaggcaagacagtcgaacgaatgagcctgagtacattgaacccaaagttctgaaacctaggaacagctacatcttgttccgaaatgcaacatccagatgctctcagagcattgatcccaactcagcatttgtttcgagggtctccagttatatctggggctcgggaatccccaatcctattgttcggcgatggttcaagtatatgggtttcttcgaagccctataccacgaagtcgactatcctgagtatatcccgaacaaactgcccaagtccccaaagaagccgaaggtccagaagggtgcaactaagtctaagaaaaagggagcaaagggtaagaacaaggttactgctctccaagtactggtcggccctaccgttggtgtcgggggaaggatgactgttagcccaatgactgctttctttagcaacaactctcacgttacgtgctatgaccccaactttgttctcgatacacctactcgagaattcctcatgatgcccctggatgatatcactctcccatcgcaaggaccaagatcagattcacaggagcagcacgtccctcggcagcctcccgatgggcaggactattttgacattctggatattgatgatttcgtttctccttatgatgacttgactactcgctcgttcaccaacaggcagctttttaagcatgccacactgggttga

SEQ ID NO: 5 - DNA, 909 bp, Yarrowia lipolytica
atgaggcaagatgagtatcatataaagcaaaggattttttcaatctcaaaagcagctataacaatgatcaagataaaaaccgagcacaaagtggtgaaggcaagaaccccgaaaaaaatgaaacctggaatggcccgtttcagaattcaagccgttcccatcatcaaccaaaacaacaacgcctacctgtcagagatcagcttcgaagaaaagttagaaccagttccgccgatgtatcctgaactacaaaaagtccttgattacattcactcacgcaaagtccccttcgacggatcatcggggccataccctccctcgagcagacgacaagtggtcaacggttatgtcgcatttaagctatatcaccacgttccacacactgaccttagcgcaattgatatcacccacctcctccaaggtccttggagagcttgccctgatagacacatctggaccaagtacgctgatcactaccgttctagagggcgtgacgtcgcattcaagacatggcttctgtctgttactagtcctgctcctgagaaggggctaattattcagcctcaaactcagagcgagcaggggtacaacttctcatcttcctctgatttttcaagttctccagaacacagtgaaacactccctgtgtcatctggagagcccagtacacctgttgaccccttcctgatcgagtttgatcaaggcatcgcggagaaagaggccagcttcacaagtccttgcaaccaaaactatgacttggatttcagccagcttcagcctctaagtattcgcgtcccttattttaaccagtacgcttcttccagtaactctggattggatgttgactactttgacggtagcaccctagacactcctagctacatcgacactttcatcacaccattgcactag

SEQ ID NO: 6 - DNA, 1104 bp, Yarrowia lipolytica
atgaaggaaaccagagatcttctcttctacaacgagaactccatcgacttcaacttctctccagaagagatggacacccatcaaatgatgtgtgtcaacaatgacagccaacaggacacatcgttcctcacagctactacttgcatttttgacttccagaaggacatctatgacgaccaaaacctgatgtccttcgaccccttctttccacagcagcgagatatggctttgtggattgcttcagtgggaaatggggctatcgattggagtactaaacctcaactgactctttcaaagaagatccctgacgtcgtcaacgaaaaaggagaggagataacccctgctgacctacaacgccactatgccgataagcatgtcgccaaacttccagatcattcctgcgatctttactaccttctattcaagcatgccaaattcgaggctaccaccctgaggatcccattgtcactcctcaatgacaacggtacaggagcacttggcccattcgacgagatcagatcgttgacggatgaagaacaacgtcttcttactctgcttaagacaatttcacattacagctgggtcactaaacatgcgaccaaagactgtacgggccaactgcttgacctgtgcaaagagggcctccagcttgtggaatctatccaggttaacaaaactttcaacggtgtcattctgcatatcctagtcgcctactttgggagaagatctactgctctaagagatgagtttcaactgcacaagctctttcttgaacaacggcttgacattgagttggaaatcttggcccaacatggaggcgctgacgccattgattctgaaaagctgcggaaggtcaagaaactgagcgaactgtgggagtggttcaatgccatcccgtacaaccaccaccctagcgtctctcagatcgagtttatcgcttctcagatcggggagaaagtaagtttcgtgaaatcctgggttgcaaacaagcgacgaactggggccaagaagagatccaagaaaccggcaccttcgacacagattagacctgcattgaaggtattcttggagaagaagccatttgtgtctcgccaagttgtgtaa